(19) United States
(12) Patent Application Publication
(10) Pub. No.: US 2024/0050601 A1
(43) Pub. Date: Feb. 15, 2024

(54) COMPOSITIONS AND ASSOCIATED METHODS FOR SUSTAINED-RELEASE OF RADIOACTIVE AGENTS AND THEIR APPLICATIONS

(71) Applicant: Precise-Therapeutics LLC, Malden, MA (US)

(72) Inventor: Weiwei Feng, Malden, MA (US)

(21) Appl. No.: 18/259,438

(22) PCT Filed: Dec. 28, 2021
(86) PCT No.: PCT/US2021/065344

§ 371 Date: Jun. 27, 2023

Related U.S. Application Data
(60) Provisional application No. 63/131,760, filed on Dec. 29, 2020.

(51) Int. Cl. A61K 51/12; A61P 35/00; A61K 51/06

(52) U.S. Cl. CPC A61K 51/1213; A61P 35/00; A61K 51/06

(57) ABSTRACT

The present disclosure is related to compositions and processes of preparation of a radiotherapeutic hydrogel that includes anionic cytotoxic radioactive agents, cationic biopolymer or the cationic biopolymer-based NP/MP, and biopolymer hydrogel. In some embodiments, the radiotherapeutic hydrogel can be potentially applied as a new approach of SIRT as adjuvant treatment (e.g., via a local administration) to selectively kill the residual cancers.

### CROSS-REFERENCE TO RELATED APPLICATIONS

This application claims the benefit of U.S. Provisional Patent Application No. 63/131,760, filed on Dec. 29, 2020; the entirety of which is hereby incorporated by reference.

### TECHNICAL FIELD

This disclosure relates to compositions and associated methods for sustained-release of radioactive agents and their applications.

### BACKGROUND

Local or locally-advanced cancers are usually treated via surgery. Because it is not always possible to remove the tumor cells completely, adjuvant treatments, such as chemotherapy and/or radiotherapy are often prescribed. One of the post-surgery adjuvant radiotherapies to prevent local recurrence is SIRT (selective internal radiation therapy). Different terms may also be used in literature, e.g., interstitial radiotherapy, interstitial-brachytherapy, micro-brachytherapy and radioembolization.

Different from systemic and external radiation therapies, SIRT is based on delivering a high radiation dose directly to the lesions to kill tumor cells with minimal damage to healthy tissues around the target. SIRT using beta-emitting radioisotopes complexed to nanoparticles (NP), microspheres (MS) or insoluble colloids, shows potential benefits, including minimal invasive delivery, outpatient treatment, and improved survival and quality of life. For instance, radioembolization by administration of Y-90-MS, via transhepatic artery injection, has been established as a safe and efficacious treatment for both primary and metastatic liver cancers. Another example of SIRT is intratumor injection of P-32-silicon MS (P-32 BioSilicon MS in a size of 30 μm), which is currently in late phase clinical trial for the treatment of pancreatic cancer. However, the efficacy of SIRT is widely variable. For instance, a survival benefit was reported for patients with head and neck cancer being treated with CCP P-32 (colloidal chromic phosphate), as well as for patients with pancreas carcinomas being treated with P-32 BioSilicon MS. On the other hand, no benefit was observed in a study of CCP P-32 treatment in 30 pancreas cancer patients. It is well known that the efficacy of SIRT using beta-emitting radioisotopes, e.g., Y-90 or P-32, complexed MS or colloidal, can be impacted by multiple factors. Among those, the most critical is the poor distribution of the radioactivity within the lesion owing to the inherit characters of the particle, and any “missed” parts of the tumor can result in residual or locally recurrent disease. Details can be found, e.g., in Bakker R. et al., Intratumoral treatment with radioactive beta-emitting microparticles: a systemic review. J Radiat Oncol 2017; 6:323; and Zhu J. L. et al., Controlling injectability and in vivo stability of thermogelling copolymers for delivery of yttrium-90 through intra-tumoral injection for potential brachytherapy. Biomaterials 2018; 180:163-72; each of which is incorporated herein by reference in its entirety.

The impact of hydrogel formulation on the distribution/retention of radioactive MSs has been recently investigated in preclinical studies. Moreover, the usage of Ho-166/chitosan hydrogel (radioactive metal chelated by chitosan, Milican), via intratumor administration, has been used for treatment of patients with small tumors, diameter not greater than 3 cm, for hepatocellular carcinoma. The issue of poor distribution also occurs in the usage of radioisotopes complexed in MS or insoluble colloid, locally administered into cavity, for example, intraperitoneal administration of CCP P-32 for the treatment of disseminated ovarian cancer. Therefore, there is a need for a new approach of SIRT with an even distribution and/or continuous delivery via infiltration within the lesion after local administration, to improve the efficacy in killing residual tumor cells with low toxicity to the surrounding normal tissues.

### SUMMARY

The present disclosure relates to cationic biopolymers for the association of anionic cytotoxic radioactive agents; processes for the preparations of the radioactive agents associate to cationic biopolymers and/or cationic biopolymer-based nanoparticles (NP) or microspheres (MS); and methods of preparations of injectable or implantable hydrogel. Specifically, this disclosure relates to a radiotherapeutic hydrogel, which combines hydrogel, cationic biopolymer-based NP/MS and anionic radioactive agents. More specifically, this disclosure describes a radiotherapeutic hydrogel specifically formulated to properly control the release rate for “sustained-release” of the anionic radioactive agent, which possesses high cytotoxicity and selectivity to proliferating tumor cells.

In accordance with one aspect of the present disclosure, the radiotherapeutic hydrogel provides physical ionizing irradiation plus “sustained-release” of the cytotoxic radioactive agent to extend the therapeutic efficacy via its infiltration to and accumulation by the surrounding proliferating tumor cells. In some embodiments, this disclosure can be used as a new approach of SIRT with efficacious eradication of residual tumors via local administration of intratumor or intracavity, for example, but not limited to, peritoneal, thoracic and postoperative surgical cavities.

In one aspect, the disclosure is related to a radiotherapeutic hydrogel, comprising: a) a biopolymer hydrogel; b) an anionic radioactive agent; and c) a cationic biopolymer or a cationic biopolymer-based nanoparticles (NP) or microspheres (MS); in some embodiments, the cationic biopolymer or the cationic biopolymer-based NP or MS associated with the anionic radioactive agent and are dispersed and/or immobilized in the hydrogel. In some embodiments, the biopolymer hydrogel is injectable or implantable. In some embodiments, the radiotherapeutic hydrogel is capable of sustainably releasing the anionic radioactive agent. In some embodiments, the radiotherapeutic hydrogel can be used for selective internal radiation therapy (SIRT) via local (e.g., intratumor or intracavity) administration. In some embodiments, the biopolymer hydrogel comprises Pluronic™ F-127, methylcellulose and/or chitosan. In some embodiments, the biopolymer hydrogel further comprises a thermosensitive polymer, a pH-sensitive polymer, a crosslinker, or a combination thereof. In some embodiments, the biopolymer hydrogel further comprises a radiolysis stabilizer. In some embodiments, the radiotherapeutic hydrogel comprises a cationic biopolymer-based NP or MS. In some embodiments, the cationic biopolymer-based NP or MS is synthesized by crosslinking cationic biopolymers (e.g., chitosan) using an anionic polymer, an ionic crosslinker, a covalent crosslinker, or a combination thereof. In some embodiments, the anionic radioactive agent is selected from: a) phosphate P-32 (e.g., H₃PO₄, H₂PO₄⁻, HPO₄²⁻, or PO₄³⁻), ATP P-32 (adenosine-5′-triphosphate), IUdR I-125 (5-iodo-2-deoxyuridine); b) anionic radioisotopes (e.g., Astatine-211, Iodine-125, or Iodine-131); and c) anionic forms of chelate-radiometal compounds. In some embodiments, the anionic radioactive agent possesses high cytotoxicity and/or selectivity to proliferating tumor cells. In some embodiments, the radiotherapeutic hydrogel is used for SIRT via an intratumor administration. In some embodiments, the intratumor administration comprises imaging-guided percutaneous and/or intraoperative injection. In some embodiments, the radiotherapeutic hydrogel is used for SIRT via an intracavity administration. In some embodiments, the intracavity administration comprises administering into peritoneal cavity, thoracic cavity, a postsurgical cavity, and/or a rescission site of a solid tumor, via a catheter impregnation and/or a direct injection.

In one aspect, the disclosure is related to a method of making a radiotherapeutic hydrogel, comprising: a) associating an anionic radioactive agent with a cationic biopolymer, forming a radioactive biopolymer; and b) dispersing and/or immobilizing the radioactive biopolymer into a biopolymer hydrogel.

In one aspect, the disclosure is related to a method of making a radiotherapeutic hydrogel, comprising: a) encapsulating an anionic radioactive agent into a cationic biopolymer-based nanoparticle (NP) or microsphere (MS), forming a radioactive biopolymer-based NP or MS; and b) dispersing and/or immobilizing the radioactive biopolymer-based NP or MS into a biopolymer hydrogel.

In some embodiments, the method further comprises formulating the biopolymer hydrogel for injection and/or implantation. In some embodiments, the anionic radioactive agent is selected from: a) phosphate P-32 (e.g., H₃PO₄, H₂PO₄⁻, HPO₄²⁻, or PO₄³⁻), ATP P-32 (adenosine-5′-triphosphate), IUdR I-125 (5-iodo-2-deoxyuridine); b) anionic radioisotopes (e.g., Astatine-211, Iodine-125, or Iodine-131); and c) anionic forms of chelate-radiometal compounds. In some embodiments, the anionic radioactive agent (e.g., phosphate P-32) possesses high cytotoxicity and/or selectivity to proliferating tumor cells. In some embodiments, the cationic biopolymer-based NP or MS is synthesized by crosslinking cationic biopolymers (e.g., chitosan) using an anionic polymer, a crosslinker, or a combination thereof. In some embodiments, the crosslinker is an ionic crosslinker, a covalent crosslinker, or a combination thereof. In some embodiments, the biopolymer hydrogel comprises Pluronic™ F-127, methylcellulose and/or chitosan. In some embodiments, the biopolymer hydrogel further comprises a thermosensitive polymer, a pH-sensitive polymer, a crosslinker, or a combination thereof, for properly controlling the gelation of the hydrogel at near physiological pH, e.g., pH 7.2±0.2, and temperature, e.g., 36.5±1.0° C. In some embodiments, the biopolymer hydrogel further comprises a radiolysis stabilizer.

In one aspect, the disclosure is related to a method for treating a subject having cancer, the method comprising administering a therapeutically effective amount of the radiotherapeutic hydrogel as described herein to the subject. In some embodiments, the subject has a solid tumor. In some embodiments, the radiotherapeutic hydrogel as a new approach of selective internal radiation therapy (SIRT) is administered via local (e.g., intratumor or intracavity) administration. In some embodiments, the radiotherapeutic hydrogel is administered through an intratumor administration. In some embodiments, the intratumor administration comprises imaging-guided percutaneous and/or intraoperative injection. In some embodiments, the radiotherapeutic hydrogel is administered into peritoneal cavity, thoracic cavity, a postsurgical cavity, and/or a rescission site of a solid tumor, via a catheter impregnation and/or a direct injection. In some embodiments, the radiotherapeutic hydrogel is biodegradable in vivo over time. In some embodiments, the radiotherapeutic hydrogel sustainably releases the anionic radioactive agent. In some embodiments, the sustainably released anionic radioactive agent effectively eradicates the satellite tumor cells surrounding the loading sites. In some embodiments, the anionic radioactive agent (e.g., phosphate P-32) possesses high cytotoxicity and/or selectivity to proliferating tumor cells. In some embodiments, the radioactivity retained within the administrated hydrogel delivers localized radiotherapy as ionizing irradiation to the region of interest as micro-brachytherapy. In some embodiments, the biopolymer hydrogel comprises Pluronic™ F-127, methylcellulose and/or chitosan. In some embodiments, the radiotherapeutic hydrogel comprises the cationic biopolymer-based NP or MS associated with the anionic radioactive agent that is dispersed into the hydrogel, in some embodiments, the cationic biopolymer-based NP or MS is synthesized by crosslinking cationic biopolymers (e.g., chitosan) using an anionic polymer, an ionic crosslinker, a covalent crosslinker, or a combination thereof.

In one aspect, the disclosure is related to a radiotherapeutic hydrogel composed for “sustained-release” of an anionic radioactive agent; wherein the radiotherapeutic hydrogel being composed as a new approach of selective internal radiation therapy (SIRT) via a local administration of intratumor or intracavity; wherein the radiotherapeutic hydrogel composing: (a) an injectable or implantable biopolymer hydrogel; (b) at least one anionic radioactive agent; and (c) a cationic biopolymer or the cationic biopolymer-based NP/MS for the association of the anionic radioactive agent, wherein the cationic biopolymer or the cationic biopolymer-based NP/MS associated with the anionic radioactive agent is dispersed and immobilized into the hydrogel.

In some embodiments, the hydrogel comprises biopolymers of, e.g., including Pluronic™ F-127, MC, CH.

In some embodiments, the biopolymer hydrogel is composed by addition of thermosensitive and/or pH sensitive polymer and/or crosslinker and combinations thereof, for a proper gelation of the hydrogel at near physiological pH and temperature.

In some embodiments, the biopolymer hydrogel is further composed with radiolysis stabilizer.

In some embodiments, the cationic biopolymer-based NP/MS associated anionic radioactive agent is dispersed; wherein the cationic biopolymer-based NP/MS is composed by crosslinking in either ionic or covalent or both crosslinkers to the cationic biopolymers, e.g., including CH.

In some embodiments, the anionic radioactive agent is one of: (a) phosphate P-32 (H₃PO₄, H₂PO₄, HPO₄²⁻, PO₄³⁻), APT P-32 (adenosine-5′-triphosphate), IUdR I-125 (5-iodo-2-deoxyuridine); (b) anionic radioisotopes, for example, but not limited to, At-211, I-125, I-131; and (c) anionic forms of chelate-radiometal compounds.

In some embodiments, the anionic radioactive agent (e.g., phosphate P-32) possesses high cytotoxicity and selectivity to proliferating tumor cells, as its involvement in their metabolism.

In some embodiments, the radiotherapeutic hydrogel as described herein is used as a new approach of SIRT via an intratumor administration, which includes imaging-guided percutaneous or intraoperative injection.

In some embodiments, the radiotherapeutic hydrogel as described herein is used as a new approach of SIRT via an intracavity administration, which includes peritoneal or thoracic cavity, or postsurgical cavity, a rescission site of a solid tumor, via a catheter impregnation or a direct injection.

In one aspect, the disclosure is related to a method of making a radiotherapeutic hydrogel for “sustained-release” of the anionic radioactive agent; the method of making the radiotherapeutic hydrogel used as a new approach of SIRT via a local administration of intratumor or intracavity; wherein the method of radiotherapeutic hydrogel composing: (a) associating and encapsulating the anionic radioactive agent into a cationic biopolymer or the cationic biopolymer-based NP/MS; (b) dispersing and immobilizing the radioactive biopolymer-based NP/MS into the hydrogel; and (c) composing biopolymer hydrogel for injection and/or implantation.

In some embodiments, the anionic radioactive agent is selected from (a) phosphate P-32 (H₃PO₄, H₂PO₄⁻, HPO₄²⁻, PO₄³⁻), APT P-32 (adenosine-5′-triphosphate), IUdR I-125 (5-iodo-2-deoxyuridine); (b) anionic radioisotopes, for example, but not limited to At-211, I-125, I-131; and (c) anionic forms of chelate-radiometals.

In some embodiments, the anionic radioactive agent (e.g., phosphate P-32) possesses high cytotoxicity and selectivity to proliferating tumor cells, as its involvement in their metabolism.

In some embodiments, the cationic biopolymer-based NP/MS is composed by crosslinking the cationic biopolymer with anionic polymer or crosslinker or combinations thereof, for the association of anionic radioactive agent; wherein the cationic biopolymer-based NP/MS being composed by crosslinking in either ionic or covalent or both crosslinkers with cationic biopolymers, e.g., including CH.

In some embodiments, the biopolymer hydrogel comprises biopolymers of, e.g., including Pluronic™ F-127, CM and CH.

In some embodiments, the biopolymer hydrogel being composed by adding thermosensitive and/or pH sensitive polymer and/or crosslinker or combinations thereof, for properly controlling the gelation of the hydrogel at near physiological pH and temperature.

In some embodiments, the hydrogel being further composed with a radiolysis stabilizer.

In some embodiments, the radiotherapeutic hydrogel is injectable and/or implantable.

In one aspect, the disclosure is related to a method used as a new approach of SIRT via a local administration of the radiotherapeutic hydrogel; wherein a method of local injection, e.g., including intratumor or intracavity injection; wherein the intratumor injection includes imaging-guided percutaneous or intraoperative injection; wherein the intracavity injection includes peritoneal or thoracic cavities, or postsurgical cavity, a rescission site of a solid tumor, via a catheter impregnation or a direct needle injection; wherein the radiotherapeutic hydrogel composing: (a) at least one anionic radioactive agent; (b) cationic biopolymer or the cationic biopolymer-based NP/MS for the association of the anionic radioactive agent; and (c) an injectable or implantable hydrogel; wherein the cationic biopolymer or the cationic biopolymer-based NP/MS associated with the anionic radioactive agent is dispersed and immobilized in the hydrogel; wherein the radiotherapeutic hydrogel is biodegradable in vivo over time, no need for surgical removal from the loading site; wherein the radiotherapeutic hydrogel “sustained release” the anionic radioactive agent; and wherein the anionic radioactive agent (e.g., phosphate P-32) possesses high cytotoxicity and selectivity to proliferating tumor cells, as its involvement in their metabolism.

In some embodiments, the radioactivity retained within the administrated hydrogel delivers localized radiotherapy as ionizing irradiation to the region of interest as micro-brachytherapy.

In some embodiments, the “sustained-release” of the anionic radioactive agents (e.g., phosphate P-32) possess high cytotoxicity and selectivity to proliferating tumor cells.

In some embodiments, the “sustained-release” of radioactive agent extends its approaching to and efficaciously eradicate the satellite tumor cells surround the loading sites.

In some embodiments, the radiotherapeutic hydrogel comprises biopolymers of, e.g., including Pluronic™ F-127, CM and CH.

In some embodiments, the cationic biopolymer-based NP/MS is composed by crosslinking the cationic biopolymer with anionic polymer or crosslinker or combinations thereof, for the association of anionic radioactive agent; the cationic biopolymer-based NP/MS being composed by crosslinking in either ionic or covalent or both crosslinkers with cationic biopolymers, e.g., including CH.

In one aspect, the disclosure is related to a method of kit for producing the radiotherapeutic hydrogel, comprising: (a) a container containing sterile hydrogel ready for dispersing radioactive CH-based NP/MS; (b) a container containing the sterile cationic biopolymer CH-based NP/MS for composing the anionic radioactive agent; and (c) instructions for preparing the radiotherapeutic hydrogel. In some embodiments, the kit further comprises a container containing a solvent for dissolving the radiotherapeutic hydrogel.

As used herein, the term “sustained-release” or “sustainable release” refers to the release of a specific agent (e.g., a radioactive agent) at a controlled rate that leads to drug delivery for a prolonged period of time. In some embodiments, the agent can be released for over 1, 2, 3, 4, 5, 6, 7, 8, 9, 10, or more than 10 days. In some embodiments, the agent can be released for over 1, 2, 3, 4, 5, 6, 7, 8, 9, 10, or more than 10 weeks.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Schematic diagram of radiotherapeutic hydrogel: chemical formula of phosphate P-32, an example of anionic radioactive agent, associated by cationic biopolymer-based nanoparticles (NP), CH-based NP with covalent crosslinker of ECH as an example, which are dispersed and immobilized within the hydrogel.

FIG. 2: Model of double-strand break after decay of the DNA incorporated phosphate P-32: a. DNA phosphate-ribose backbone; b. phosphate P-32 DNA incorporation; c. double-strand break: the strand can be chemically broken after decay of P-32 to S-32, and the beta-particle radiation can break the other strand due to the short distance (about 2 nm) between double helix. Contrast to DNA incorporated P-32, there is only a small chance for a beta-particle emitted from radioisotope to travel in a precise orientation causing the double-strand break.

FIGS. 3A-3B: Assessment of the impact of CH:TPP ratio on the size of CH/TPP NPs. In this experiment, CH/TPP NP was created in a final concentration of 1 mg/ml CH, a reaction pH of approximately 4. An average NP size of 30 nm or 170 nm was obtained with a CH:TPP ratio (w/w) of 3:1 (top) or 2.5:1 (bottom), respectively.

FIGS. 4A-4B: Assessment of the impact of CH molecular weight on the size of CH/ECH NPs. Wherein, the CH/ECH NP was synthesized with the amine groups inside CH being protected by TPP prior to ECH covalent crosslinking, and with a final removal of TPP from CH/TPP/ECH NPs. An average NP size of 512 nm or 927 nm was observed with a CH molecular weight of 50-190 kDa (small mw; top) or 190-310 kDa (medium mw; bottom), respectively.

FIG. 5: Viscosity of hydrogel composed of 13% Pluronic™ F127/4% MC (w/w) with (solid column) or without (open column) additional 0.5% CH (w/w) over different temperature.

FIG. 6: In vitro stability of hydrogel of 13% Pluronic™ F127/4% MC/0.5% CH (w/w) within PBS (pH 7.2; solid circle) or 0.1 M citrate buffer (pH 5.5; open circle) over 3 weeks incubation at 37° C.

FIG. 7: Optical imaging of hydrogel 13% F127/4% MC containing 0.2 mg/ml ICG immediately (0-h), and at 1-h, 2-h, l-d, 2-d, 4-d & 8-d post injection (subcutaneous, 500 μl) in healthy Balb/C mice (n=4).

FIG. 8: Optical imaging of hydrogel 13% F127/4% MC containing 0.2 mg/ml ICG immediately (0), and at 1, 2, 4, 6, 24 & 48-hours post injection (intramuscular, 50 μl) in healthy Balb/C mice (n=3).

### DETAILED DESCRIPTION

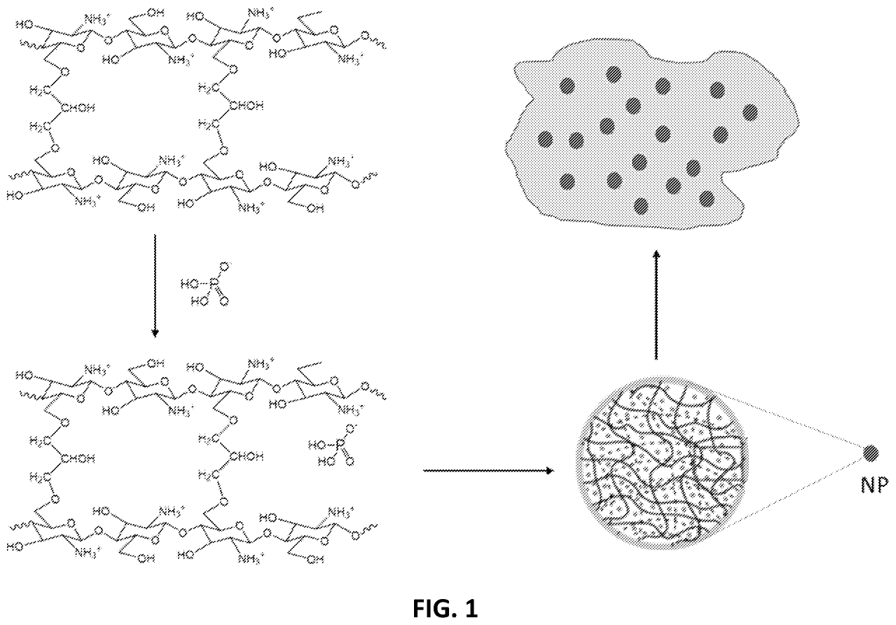

The present disclosure describes compositions and processes of preparation for injectable or implantable biopolymer hydrogel, wherein dispersed and immobilized with anionic radioactive agents associated cationic biopolymer-based NP/MS (FIG. 1). Specifically, the disclosure describes a radiotherapeutic hydrogel, which is thermoreversibly sensitive, pliable and cavity adjusting, and solidifies at body temperature after administration. More specifically, this disclosure relates to a radiotherapeutic hydrogel specifically formulated for properly controlling the rate of “sustained-release” of the anionic radioactive agents. In one embodiment, in addition to its effect of physical ionizing irradiation as SIRT or micro-brachytherapy, the present disclosure relates to “sustained release” of the radioactive agents which possess high cytotoxicity and selectivity to proliferating cancer cells, to efficaciously eradicate the surrounding satellite tumor cells via its extended infiltration post a local administration.

The present disclosure is related to a radiotherapeutic hydrogel that includes anionic cytotoxic radioactive agents, cationic biopolymer or the cationic biopolymer-based NP/MP, and biopolymer hydrogel. In some embodiments, the radiotherapeutic hydrogel can be potentially applied as a new approach of SIRT as adjuvant treatment via a local administration to selectively kill the residual cancers. This disclosure relates to processes for the preparations of a cationic biopolymer or the cationic biopolymer-based NP/MS and its association with the anionic radioactive agent, and to the methods of hydrogel formulations. Specifically, this disclosure describes hydrogel, wherein dispersed and immobilized with anionic radioactive agents associated cationic biopolymer-based NP/MS. More specifically, this disclosure of radiotherapeutic hydrogel is specifically formulated for properly controlling the rate of “sustained-release” of the radioactive therapeutic agents within the lesion. In one embodiment, this disclosure includes a local administration of intratumor or intracavity, for example, but not limited to peritoneal, thoracic and postoperative surgical cavities. As a potential new approach of SIRT, this disclosure includes two aspects after local administration: 1) physical ionizing irradiation as micro-brachytherapy, 2) “sustained-release” of the radioactive therapeutic agent to extend via infiltration and efficaciously eradicate the proliferating cancer cells surround the loading sites with low toxicity to nearby normal tissues.

### Cationic Biopolymers

The present disclosure relates to cationic biopolymers, which are characterized as macromolecules that possess positive charges, either inherent in polymer side chains and/or its backbone. Cationic biopolymers have been employed commonly in drug delivery because of their superior encapsulation efficiency, low toxicity, and improved profile. This disclosure relates to natural or synthetic cationic polymers, including, but not limited to cationic peptides and their derivatives (e.g., polylysine and polyornithine), linear or branched synthetic polymers (e.g., polybrene and polyethyleneimine), polysaccharides (e.g., cyclodextrin, cellulose, and chitosan), natural polymers (e.g., histone, collagen, and gelatin), and activated and non-activated dendrimers.

Chitosan (CH), an N-deacetylated product of chitin, as an example, has been proved as a biopolymer with properties of low-toxicity, biodegradability, biocompatibility, hemostasis and biomembrane-adhesion. CH and its derivatives have received significant scientific interests and have become one of the hottest topics in recent decades, especially for medical and pharmaceutical applications, including in drug delivery. CH-based NPs can efficiently remove anionic nutrients, including orthophosphate, in wastewater treatment. In addition, phosphate-32/CH hydrogel coated on the surface of poly(ethylene terephthalate) balloon is under study for potential usage as brachytherapy in the treatment of coronary restenosis. Moreover, owing to its cationic property that can easily bind to negatively charged biomembrane surfaces as well as the capability of penetrating the tight junctions between epithelial cells, CH is a good candidate for the drug encapsulation and controlled-release. CH-based hydrogel for potential prevention of postoperative peritoneal adhesion is under investigation by numerous groups.

Low toxicity of CH: CH is widely regarded as being a non-toxic, biologically compatible biopolymer. It is approved for dietary applications in Japan, Italy, and Finland. CH has also been approved by the FDA for use in wound dressings. The modifications made to CH can make it more or less toxic and any residual reactants should be carefully removed.

Chitosan is found to display little in vitro cytotoxicity against CCRF-CEM (human lymphoblastic leukemia) and L132 (human embryonic lung cells) (IC₅₀>1 mg/ml). Interestingly, CH seems to be toxic to several bacteria, fungi and parasites. This pathogen-related toxicity is an effect which could aid in infectious disease control.

In vivo toxicity assessment particularly after long term administration is of high importance for CH designed for the usage in drug delivery. In a relatively long study (65 days), no detrimental effect on body weight was found when CH oligosaccharides were injected (7.1-8.6 mg/kg over 5 days). However, there was a report that a high dose of CH (50 mg/kg) with an intravenous administration caused mice death, probably due to blood aggregation. CH was not pyrogenic and caused no eye or skin irritation in rabbits and guinea pigs respectively. No toxic effect was noted after subcutaneous implantation of 200 μl of 30 mg/ml photo-crosslinked azide-CH-lactose gel. It is reported that CH has an LDs of greater than 16 g/kg in oral administration to mice, which is comparable to that of sucrose.

### Chitosan Degradation & Biodistribution

Chemical characterization assays determining the degradation of CH commonly use viscometer and/or gel permeation chromatography to evaluate a decrease in molecular weight. CH is stable in vitro under non-enzymatic conditions, and lysozyme has been found to efficiently degrade CH. For example, 50% acetylated CH showed a 66% loss in viscosity after a 4 hour in vitro incubation at pH 5.5 (0.1 M phosphate buffer, 0.2 M NaCl, 37° C.). This degradation appears to depend on the degree of acetylation with degradation of acetylated CH (more chitin like) showing a faster rate. A range of proteases have been found to degrade CH-formed film in varying degrees, with leucine amino-peptidase being the most effective, which can degrade the film by 38% over 30 days. The type of crosslinker used for the film formation can also influence the degradation rate, glutaraldehyde to a greater degree than tripolyphosphate, in an effect that is more pronounced for CH in high (310-600 kDa) and medium (190-310 kDa) molecular weight.

Chitosan degradation after intravenous administration has been rarely reported because of the potential blood agglutination. It is somewhat unclear what the mechanism of degradation is when CH is injected intravenously. It is likely that degradation and elimination of CH are strongly dependent on the molecular weight and the degree of acetylation. Possible sites of degradation, inferred from the localization of CH, may be in liver and kidney. CH has also been administered subcutaneously, inmost cases as an implant. A proposed skin substitute of glutaraldehyde cross-linked CH/collagen is relatively stable over time compared to collagen alone when implanted subcutaneously in rabbits.

The distribution of CH is related to all aspects of its formulation from the molecular weight and degree of deacetylation to the nanoparticle size. In the case of a nanoparticulate formulation, the kinetics and biodistribution are initially controlled by the size and charge of the NPs, but not by CH. However, after particle decomposition to CH, free CH can distribute in the body and eliminate thereafter. Elimination processes can be preceded by biodegradation. Obviously, CH can be modified in such a way that it is possible to create formulations that exhibit quick release characteristics or long-acting dosage forms which therefore affect CH kinetics, metabolism and excretion.

### Anionic Cytotoxic Radioactive Agents

This disclosure describes the usage of anionic and cytotoxic radioactive agents of, for example, but not limited to, phosphate P-32 (e.g., H₃PO₄, H₂PO₄⁻, HPO₄²⁻ PO₄³⁻, P-32), APT P-32 (e.g., adenosine-5′-triphosphate), IUdR I-125 (e.g., 5-iodo-2-deoxyuridine); anionic radioisotopes, for example, but not limited to, At-211, I-125, I-131, as well as anionic forms of chelate-radiometal compounds. The cytotoxic radioactive agents described here are group of radioactive agents as their potential involvement in the metabolism of proliferating cells, such as tumor cells.

An example of anionic and cytotoxic radioactive agents is phosphate P-32. Phosphorus-32 is a pure beta-emitting radioisotope (T_{1/2}=14.3 days, Eₘₐₓ=1.71 MeV), and decays into non-radioactive surfur-32. The maximum penetration range of a P-32 beta particle in soft tissue is 0.8 cm; while, approximately 50% of the energy is absorbed in the first 0.1 cm. Phosphate P-32 (8.5˜9.0 Ci/mmole) has been widely used for the treatment of myeloproliferative neoplasms (MPNs) as well as for palliation of pain arising from osseous metastases for over eighty years since it was first used in human in 1938. However, owing to the concern over its leukemogenic potential and availability of other therapeutic modalities for chronic leukemias, phosphate P-32 is now mainly used only in the treatment of polycythemia rubra vera (PRV) and essential thrombocythemia (ET). Details can be found, e.g., in Tennvall J. & Brans B. EANM procedure guideline for 32P phosphate treatment of myeloproliferative diseases. Eur J Nucl Med Mol Imaging 2007; 34:1324-7; McMullin M. F. et al. Guidelines for the diagnosis, investigation and management of polycythemia/erythrocytosis. Brit J Haematol. 2005; 130:174-195; and Harrison C., Bareford D., Butt N. et al. Guidelines for the investigation and management of adults and children presenting with thrombocytosis. Brit J Haematol. 2010; 149:352-375; each of which is incorporated herein by reference in its entirety. On the other hand, except the leukemogenic potential with high dose, data over the eighty years has demonstrated that phosphate P-32 in a low dose range (5-7 mCi per intravenous injection, total up to 4 injections, if necessary) is a well-tolerated and efficacious treatment option in MPNs.

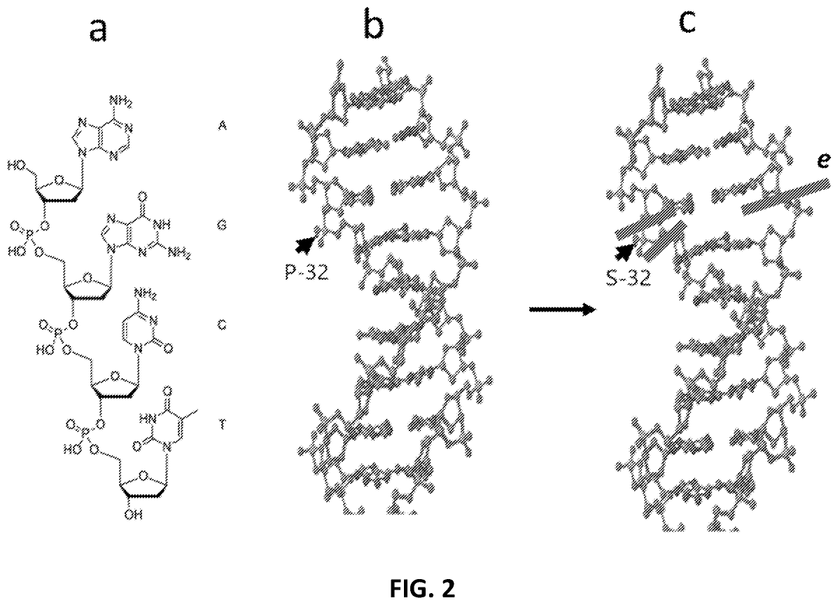

Phosphate P-32, as compared to other beta-emitting radioisotopes/compounds, is extremely cytotoxic to proliferating cancer cells owing to its unique chemical and radiobiological properties. Phosphate P-32 is a substance for DNA synthesis in cell proliferation, and the DNA incorporated phosphate P-32 leads to a high possibility of double-strand breakage after its decay from P-32 to S-32 (FIG. 2). Details can be found, e.g., in Cheng Y. et al., Phosphoruse-32, a clinically available drug, inhibits cancer growth by inducing DNA double-strand breakage. PLOS One 2015; 0128152, which is incorporated herein by reference in its entirety. In this schematic, P-32 is incorporated directly into one strand of replicating DNA. The physical decay of P-32 to S-32 causes chemical breakage of the DNA strand; in addition, the beta particles released from this radioactive decay event needs to travel only 2 nm to reach the contralateral strand of the double helix, severing it and thus causing a double-strand break at this genomic locus. This mechanism stands in stark contrast to non-incorporated beta-emitting radioisotopes, where only a very small fraction of emitted beta particles travels in the precise orientation necessary to strike one strand plus its opposite strand and cause a double-strand DNA break. With DNA incorporated P-32, the extreme proximity of the contralateral target strand to the decay-produced beta particle makes this double-strand breakage much more likely to occur.

Over the concerns of its high systemic toxicity, e.g., bone marrow, phosphate P-32 has never been established as a primary anticancer strategy for solid tumors. Details can be found, e.g., in Tennvall J. & Brans B. EANM procedure guideline for 32P phosphate treatment of myeloproliferative diseases. Eur J Nucl Med Mol Imaging 2007:34:1324-7. However, this disclosure applies the advantages of its high cytotoxicity and selectivity to proliferating tumor cells with a minimal systemic exposure of phosphate P-32. A brief outline of the advantages of this disclosure being potentially used as a new approach of SIRT, micro-brachytherapy plus “sustained-release”, is listed as follows: First, phosphate P-32 is an FDA-approved drug with a known and tolerated toxicity profile. Second, phosphate P-32 is proved as “tumor-seeking” and preferentially being absorbed by proliferating tumor cells. Third, its mechanism of favorable DNA double-strand breakage as the strand can be chemically broken after the DNA incorporated P-32 decayed to surfur-32 (S-32, non-radioactive), in addition to its radiobiological property. Fourth, most importantly, the character of “sustained-release” of the present disclosure increases the concentration, retention and infiltration of phosphate P-32 within the lesion with a low systemic exposure. Hence, this disclosure of radiotherapeutic hydrogel, wherein dispersed and immobilized phosphate P-32 associated cationic biopolymers and/or cationic biopolymer-based NP/MS, possesses advantages of phosphate P-32 in efficacious eradication of residual tumors with decreased side effects by limited systemic exposure.

A proper rate of “sustained-release” can be the one equivalent to the physical decay half-life of the associated radioisotope, owing to the potential fast clearance of the released free radioactive agents from the target region. For this disclosure, these two concepts can be applied for the retention or slowing the “sustained-release” of the radioactive agents from the implant: the association of the radioactive agent in CH-based NP/MS and hydrogel environment of the implant. Also, there are two concepts that can apply for the rate of “sustained-release”, the degradation of the hydrogel and the dissociation of the radioactive agent from the NP/MS/hydrogel implants.

In some embodiments, this disclosure of the hydrogel formulated for properly controlling the rate of “sustained-release” can also be used for non-radioactive therapeutic agents. These therapeutic agents include, but are not limited to, anionic form of folic acid, oligonucleotide, DNA or RNA for cancer targeted therapy or cancer vaccine.

### Preparation of Hydrogel Dispersed and Immobilized with Radioactive NP/MS

The present disclosure describes the processes of anionic radioactive agents' association into cationic biopolymers and/or into cationic biopolymer-based NP/MS, which then being dispersed and immobilized in hydrogel. In addition, this disclosure describes a hydrogel, which can generate a pliable and cavity adjusting, and being solidified gelation under body temperature after local administration. The hydrogel is formed by biopolymer(s) with or without addition of surfactants/crosslinkers. The gelation of the injectable hydrogel can be in-situ formed post administration by physical stimulations, for example, but not limited to the change of temperature, pH, or solvent exchange. Moreover, the viscosity of hydrogel can be adjusted according the potential application, e.g., can be in liquid or semi-solid forms under ambient temperature for injection or implantation, respectively.

### Synthesis of Cationic Biopolymer-Based NP/MS

The present disclosure describes synthetic methods of cationic biopolymer-based NP/MS formed either directly or by addition of crosslinkers. The network of the cationic biopolymer-based NP/MS with crosslinkers can be formed either ionically or covalently. Three different groups of anionic crosslinkers have been assessed in this disclosure, e.g., small molecular ionic crosslinkers, anionic polymers, and small molecular covalent crosslinkers. Specifically, the small molecular ionic crosslinker includes, but not limited to, citrate and tripolyphosphate (TPP); Anionic polymer includes, but not limited to alginate, hyaluronic acid (HA), carrageenan, carboxymethylcellulose (CMC), polyglutamic acid; small molecular covalent crosslinker includes, but not limited to, glutaraldehyde (GA), epichlorohydrin (ECH) and ethylene glycol diglycidyl ether (EGDGE). In order to properly control the rate of “sustained-release” of anionic radioactive agents, the size, surface charge and strength of the formed NP/MS have been assessed and optimized.

Nanoparticles (NP) described in this disclosure are particles with a size range of 10 to 1000 nm in diameter, more specifically, a size range of 50 to 500 nm (e.g., 50-450 nm, 50-400 nm, 50-350 nm, 50-300 nm, 50-250 nm, 50-200 nm, 50-150 nm, 50-100 nm, 100-500 nm, 100-450 nm, 100-400 nm, 100-350 nm, 100-300 nm, 100-250 nm, 100-200 nm, 100-150 nm, 150-500 nm, 150-450 nm, 150-400 nm, 150-350 nm, 150-300 nm, 150-250 nm, 150-200 nm, 200-500 nm, 200-450 nm, 200-400 nm, 200-350 nm, 200-300 nm, 200-250 nm, 250-500 nm 250-450 nm, 250-400 nm, 250-350 nm, 250-300 nm, 300-500 nm, 300-450 nm, 300-400 nm, 300-350 nm, 350-500 nm, 350-450 nm, 350-400 nm, 400-500 nm, 400-450 nm, or 450-500 μm); while the size of MS described here is in a range of 1 to 100 μm, more specifically, a size range of 1 to 30 μm (e.g., 1-25 μm, 1-20 μm, 1-15 μm, 1-10 μm, 1-5 μm, 5-30 μm, 5-25 μm, 5-20 μm, 5-15 μm, 5-10 μm, 10-30 μm, 10-25 μm, 10-20 μm, 10-15 μm, 15-30 μm, 15-25 μm, 15-20 μm, 20-30 μm, 20-25 μm, or 25-30 μm). Specifically, the size, surface charge and strength of CH-based NP/MS, as an example, can be impacted by multiple factors, e.g., CH characteristics such as molecular weight and degree of deacetylation (DD), with or without addition of crosslinkers and/or stabilizers. The preparation of CH-based NP/MS includes methods, for example, but not limited to these briefly described below:

Ionotropic Gelation

The method is based on electrostatic interaction between amine groups of CH and negatively charged groups of polyanion such as tripolyphosphate (TPP). The anionic phosphate group of TPP interacts with the positively charged amine group inside CH thereby stabilizing the NPs. In brief, for example, solutions CH and TPP in proper concentrations are prepared by dissolving in 0.1 M acetic acid and distilled water, respectively. More specifically, the concentration range tested here are in a range of 0.2 to 10 mg/ml and 0.1 to 5 mg/ml for CH and TPP, respectively. TPP solution is added dropwise into the CH solution under magnetic stirring, and kept the mixture under stirring for additional 60 minutes or overnight. The formed NP can be purified from remained non-reacted TPP by centrifuge or dialysis against water. The size, surface charge as well as the strength of particles can be adjusted by, e.g., varying the molecular weight and degree of deacetylation (DD) of CH, concentration and molar ratio of CH to TPP, as well as with the addition of stabilizers/surfactants.

Microemulsion

This is a method in which the free amino group inside CH can be conjugated with glutaraldehyde, and NP i s formed in the presence of surfactant. Particle size can be controlled by varying the amount of glutaraldehyde which changes the degree of cross-linking. Briefly, a surfactant is dissolved in N-hexane, CH in acetic solution and glutaraldehyde are then mixed to form surfactant/hexane mixture under continuous stirring at room temperature. The systems are stirred overnight to ensure the completion of the cross-linking process.

Polyelectrolyte Complex (PEC)

The mechanism of formulation of CH-based NPs by PEC is the electrostatic interactions between positive charge group inside CH and the negative charge group of anionic polymers, for example, but not limited to, alginate, carrageenan, hyaluronic acid, and carboxymethyl cellulose. Alginate, as an example, is a natural water-soluble linear polysaccharide extracted from brown seaweeds and marine algae, its carboxylic acid groups thus being able to interact electrostatically with amine groups on CH to form NP. Another example of anionic polymer for PEC is hyaluronic acid (HA), which is also known as hyaluronan or hyaluronate. HA is a natural, non-toxic, biocompatible and biodegradable polysaccharide of disaccharides composed of D-glucuronic acid and N-acetyl-D-glucosamine. HA is ubiquitously found throughout the different tissues of living organisms, especially in connective, epithelial and neural tissues. The molecular weight of HA ranges from 100 kDa in serum to 8000 kDa in vitreous. HA has been proven to be promising biomedical materials, due to their tunable sizes, colloidal stability, low cytotoxicity, protection from enzymatic degradation. HA can also be used as a polyanion coating material on the surface of positively charged CH/TPP NPs. The size of the complexed NPs via PEC can be varied from 50 to 700 nm.

Complex Coacervation

One of the examples of this method is via coacervation between the positively charged amine groups on CH and negatively charged phosphate groups on DNA, CH-DNA NPs has been reported to form readily. The CH-based NPs prepared in this manner can prevent or decrease the degradation rate of DNA post administration and then improve its bioavailability.

Solvent Evaporation

In this method, the emulsification of the polymer solution into an aqueous phase followed by the evaporation of the polymer solvent which induces the precipitation of polymer as nanospheres is required.

Coprecipitation A method in which CH-based NPs with a high degree of size uniformity can be prepared by grafting lactic acid on CH to serve as a drug carrier for prolonged drug release.

NP/WS/Phosphate P-32 Association

This disclosure describes the association methods of phosphate P-32 (P-32) into the cationic biopolymer-based NP/MS. For example, CH-based NP/MS/phosphate P-32 (CH—NP/P-32) can be prepared by compounding P-32 to CH either prior to, during or after the formation of CH-based NP/MS (CH—NP).

The formed CH-based NP/MS can be separated from any remaining start material by any suitable method known in the art. In one embodiment, the formed CH—NP can be separated from remaining start materials by adding ethanol to precipitate CH—NP, centrifuging and/or dialyzing against water. More specifically, the formed CH—NPs have been pelleted and water washed by centrifugation in a Beckman Allegra X-12 centrifuge. Dialysis is the process of separating molecules in solution by difference in their rate of diffusion through a semipermeable membrane, such as dialysis tubing. The nanoparticle size and zeta potential for the surface charge have been analyzed by Particle Analyzer Litesizer 500. As an alternative, the fraction of free amino groups in CH—NP can also be determined by the TNBS (2,4,6-trinitrobenzenesulfonic acid) assay. TNBS reacts preferentially with primary amino groups to form a chromophore readily measured by colorimetric means at 335 nm. Briefly, 0.15 mL of CH—NP sample can be mixed with an equal volume of 0.05% TNBS and then incubated at 37° C. for 3 hours. Following incubation, 0.15 mL of 10% SDS and 0.125 mL 1 M HCl can be added to terminate the reaction. A portion (0.2 mL) of the mixture is transferred to a 96-well plate and the absorbance can be measured at 335 nm. The fraction of free amino groups in each CH—NP preparation can be determined by comparing to a standard curve.

A proper strength or stability of the formed CH—NP is also critical to provide sufficient protection for the drug cargo for the “sustained-release”. To determine the stability of CH—NP structure over time, the synthesized CH—NP has been incubated either in water suspension or in acidic solution, e.g., 0.5 mM acetic acid (pH of about 4.0) over time. The size distribution of the CH—NP can be compared prior and after incubation.

The present disclosure includes evaluation, comparison and optimization of the methods for CN—NP/P-32 association. The yield and stability of CN—NP/P-32 association have been assessed. Under a certain amount of CH—NP, the impacts of pH, amount of P-32 as well as CH—NP synthesized with different crosslinkers on CN—NP/P-32 association have been assessed. In acidic reaction conditions, the amine sidechain on the CH—NP becomes cationic which acts as a natural binder to retain anions (such as phosphate), which is essentially the basis for ion-exchange chromatography. The yield of CN—NP/P-32 association can be measured by a proper assay, such as instant thin layer chromatography (iTLC) or spectrophotometric assay. The iTLC analysis can be performed with a developing solution of MeOH:H₂O:acetic acid (49:49:2). Rf value for P-32 and CN—NP/P-32 can be determined. The spectrophotometric assay has been used for the analysis for mimic reactions of CH—NP/P-32, where non-radioactive phosphate has been used in the place of P-32. In spectrophotometric assay, the CH—NP/P-32 reaction solution is centrifuged, the amount of phosphate in the supernatant is measured and compared to the total amount of phosphate added. Detail information on this assay can be found in the reference of Method 365.3: Phosphorous, all forms, United States Environmental Protection Agency, which is incorporated herein by reference in its entirety.

### Formation of Injectable or Implantable Biopolymer Hydrogel

Hydrogels can be formed by hydrophilic polymers, which contain a significant amount of water, maintaining a self-organized three-dimensional structure. Hydrogel composed with biopolymer, e.g., polysaccharides, holds great potential as desirable biomaterials in drug delivery, which requires the in situ gelling to properly control the release rate of drugs/cells in a specific location after administration. In accordance with one aspect of the present disclosure, polysaccharide-based hydrogel is described. In one embodiment, the disclosure relates a hydrogel composed with polysaccharides of methylcellulose (MC) and CH, surfactants with or without a trace amount of crosslinkers. Specifically, Pluronic™ F127 (F-127), a nonionic surfactant, exhibits gelation based on micellization above the critical micelle temperature (CMT) or critical micelle concentration (CMC) in water. The presence of biopolymers, e.g., MC, might cause an MC-assisted interconnected network of micelles, lowing CMT of Pluronic™ F127 and increasing the strength of the hydrogel. The mixture of Pluronic™ F127 and MC exhibits thermoreversible gelation upon heating, and the strength of the formed gel is dependent on the concentration and temperature. In this disclosure, the addition of a low concentration of CH in hydrogel can be helpful to increase the strength as well as to properly control the rate of “sustained-release” for cytotoxic radioactive agents. Different from poor solubility of CH, MC and its derivatives are water soluble, non-ionic and pH stable thermosensitive polysaccharides, with a gelation temperature of about 50-60° C. Moreover, to further enhance its strength/stability, hydrogel can be formed by adding a trace amount of crosslinkers, for example, but not limited to CMC, HA, alginate, genipin, GP or TPP. In general, hydrogel described here remains as liquid or semi-solid under room temperature and forms a gel in situ at near physiological temperature and pH after local administration. More specifically, this disclosure relates a polysaccharide-based hydrogel, wherein dispersed and immobilized CH—NP/P32 for the potential adjuvant treatment for locally residual tumors with a local administration.

In addition to the biopolymer-based hydrogels prepared above, the addition of radiolysis protecting agent or stabilizer (e.g., ascorbic acid, methionine, gentisic acid) can be necessary for radiolysis protection for the final product of the radiotherapeutic hydrogel.

The methods of the present disclosure can also be performed using a formulation kit, capable of being associated with anionic radioactive agents on the application site. Such kits can contain cationic polymers and/or cationic polymer-based NP/MS in sterile form and can include a sterile container of an acceptable reconstitution liquid. Such kits can also include, if desired, other conventional kit components, for example, one or more carriers, and/or one or more additional vials for mixing. Instructions, either as inserts or labels, indicating quantities of the embolic composition and carrier, guidelines for mixing these components, and protocols for administration can also be included in the kit. Sterilization of the containers and any materials included in the kit and lyophilization (also referred to as freeze-drying) of the embolic composition can be carried out using conventional sterilization and lyophilization methodologies known to those skilled in the art. Lyophilization aids useful in the formulation kits can also include, for instance, mannitol, lactose, and sorbitol. Stabilization aids useful in the formulation kits include but are not limited to ascorbic acid, cysteine, monothioglycerol, gentisic acid, and inositol.

In some embodiments, the hydrogel as described herein further comprises a “radiolysis stabilizer.” As used herein, a “radiolysis stabilizer” refers to radiolysis protecting agent, e.g., ascorbic acid, methionine, gentisic acid.

In some embodiments, the biopolymer hydrogel is composed by addition of thermosensitive and/or pH sensitive polymer and/or crosslinker and combinations thereof, for a proper gelation of the hydrogel at near physiological pH and temperature. As used herein, the term “thermosensitive polymer” refers to polymers that exhibit a drastic and discontinuous change of their physical properties with temperature. As used herein, the term “pH-sensitive polymer” refers to polymers that exhibit a drastic and discontinuous change of their physical properties with pH. In some embodiments, the gelation starts at a temperature around or above 25, 26, 27, 28, 29, 30, 31, 32, 33, 34, 35, 36, or 37° C.

In some embodiments, the disclosure is related to methods of making a radiotherapeutic hydrogel, including: a) synthesizing cationic biopolymer-based NP or MS; b) associating (e.g., encapsulating) an anionic radioactive agent (e.g., phosphate P32) with the cationic biopolymer-based NP or MS; and c) dispersing and/or immobilizing the product of step b) into a biopolymer hydrogel.

In some embodiments, the cationic biopolymer-based NP or MS is synthesized by the following steps, as an example.

Step-1, synthesizing CH-based NP by adding an ionic crosslinker (e.g., gelatin, alginate, HA, glutaraldehyde, and/or TPP) solution dropwise to a CH solution under stirring. In some embodiments, the reaction mixture is kept stirring at ambient temperature for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours. In some embodiments, the pH of the synthesized CH/TPP NP is about 3-6, e.g., about 3, about 3.5, about 4, about 4.5, about 5, about 5.5, or about 6. In some embodiments, the starting concentration of CH is about 0.5-5 mg/ml, e.g., about 0.5 mg/ml, about 1 mg/ml, about 1.5 mg/ml, about 2 mg/ml, about 2.5 mg/ml, about 3 mg/ml, about 3.5 mg/ml, about 4 mg/ml, about 4.5 mg/ml, or about 5 mg/ml. In some embodiments, the molecular weight of CH is about 10-310 kDa (e.g., about 10-300 kDa, about 10-290 kDa, about 10-280 kDa, about 10-270 kDa, about 10-260 kDa, about 10-250 kDa, about 10-240 kDa, about 10-230 kDa, about 10-220 kDa, about 10-210 kDa, about 10-200 kDa, about 10-190 kDa, about 10-180 kDa, about 10-170 kDa, about 10-160 kDa, about 10-150 kDa, about 10-140 kDa, about 10-130 kDa, about 10-120 kDa, about 10-110 kDa, about 10-100 kDa, about 10-90 kDa, about 10-80 kDa, about 10-70 kDa, about 10-60 kDa, about 10-50 kDa, about 10-40 kDa, about 10-30 kDa, about 10-20 kDa, about 20-310 kDa, about 20-300 kDa, about 20-290 kDa, about 20-280 kDa, about 20-270 kDa, about 20-260 kDa, about 20-250 kDa, about 20-240 kDa, about 20-230 kDa, about 20-220 kDa, about 20-210 kDa, about 20-200 kDa, about 20-190 kDa, about 20-180 kDa, about 20-170 kDa, about 20-160 kDa, about 20-150 kDa, about 20-140 kDa, about 20-130 kDa, about 20-120 kDa, about 20-110 kDa, about 20-100 kDa, about 20-90 kDa, about 20-80 kDa, about 20-70 kDa, about 20-60 kDa, about 20-50 kDa, about 210-40 kDa, about 20-30 kDa, about 30-310 kDa, about 30-300 kDa, about 30-290 kDa, about 30-280 kDa, about 30-270 kDa, about 30-260 kDa, about 30-250 kDa, about 30-240 kDa, about 30-230 kDa, about 30-220 kDa, about 30-210 kDa, about 30-200 kDa, about 30-190 kDa, about 30-180 kDa, about 30-170 kDa, about 30-160 kDa, about 30-150 kDa, about 30-140 kDa, about 30-130 kDa, about 30-120 kDa, about 30-110 kDa, about 30-100 kDa, about 30-90 kDa, about 30-80 kDa, about 30-70 kDa, about 30-60 kDa, about 30-50 kDa, about 30-40 kDa, about 40-310 kDa, about 40-300 kDa, about 40-290 kDa, about 40-280 kDa, about 40-270 kDa, about 40-260 kDa, about 40-250 kDa, about 40-240 kDa, about 40-230 kDa, about 40-220 kDa, about 40-210 kDa, about 40-200 kDa, about 40-190 kDa, about 40-180 kDa, about 40-170 kDa, about 40-160 kDa, about 40-150 kDa, about 40-140 kDa, about 40-130 kDa, about 40-120 kDa, about 40-110 kDa, about 40-100 kDa, about 40-90 kDa, about 40-80 kDa, about 40-70 kDa, about 40-60 kDa, about 40-50 kDa, about 50-310 kDa, about 50-300 kDa, about 50-290 kDa, about 50-280 kDa, about 50-270 kDa, about 50-260 kDa, about 50-250 kDa, about 50-240 kDa, about 50-230 kDa, about 50-220 kDa, about 50-210 kDa, about 50-200 kDa, about 50-190 kDa, about 50-180 kDa, about 50-170 kDa, about 50-160 kDa, about 50-150 kDa, about 50-140 kDa, about 50-130 kDa, about 50-120 kDa, about 50-110 kDa, about 50-100 kDa, about 50-90 kDa, about 50-80 kDa, about 50-70 kDa, about 50-60 kDa, about 60-310 kDa, about 60-300 kDa, about 60-290 kDa, about 60-280 kDa, about 60-270 kDa, about 60-260 kDa, about 60-250 kDa, about 60-240 kDa, about 60-230 kDa, about 60-220 kDa, about 60-210 kDa, about 60-200 kDa, about 60-190 kDa, about 60-180 kDa, about 60-170 kDa, about 60-160 kDa, about 60-150 kDa, about 60-140 kDa, about 60-130 kDa, about 60-120 kDa, about 60-110 kDa, about 60-100 kDa, about 60-90 kDa, about 60-80 kDa, about 60-70 kDa, about 70-310 kDa, about 70-300 kDa, about 70-290 kDa, about 70-280 kDa, about 70-270 kDa, about 70-260 kDa, about 70-250 kDa, about 70-240 kDa, about 70-230 kDa, about 70-220 kDa, about 70-210 kDa, about 70-200 kDa, about 70-190 kDa, about 70-180 kDa, about 70-170 kDa, about 70-160 kDa, about 70-150 kDa, about 70-140 kDa, about 70-130 kDa, about 70-120 kDa, about 70-110 kDa, about 70-100 kDa, about 70-90 kDa, about 70-80 kDa, about 80-310 kDa, about 80-300 kDa, about 80-290 kDa, about 80-280 kDa, about 80-270 kDa, about 80-260 kDa, about 80-250 kDa, about 80-240 kDa, about 80-230 kDa, about 80-220 kDa, about 80-210 kDa, about 80-200 kDa, about 80-190 kDa, about 80-180 kDa, about 80-170 kDa, about 80-160 kDa, about 80-150 kDa, about 80-140 kDa, about 80-130 kDa, about 80-120 kDa, about 80-110 kDa, about 80-100 kDa, about 80-90 kDa, about 90-310 kDa, about 90-300 kDa, about 90-290 kDa, about 90-280 kDa, about 90-270 kDa, about 90-260 kDa, about 90-250 kDa, about 90-240 kDa, about 90-230 kDa, about 90-220 kDa, about 90-210 kDa, about 90-200 kDa, about 90-190 kDa, about 90-180 kDa, about 90-170 kDa, about 90-160 kDa, about 90-150 kDa, about 90-140 kDa, about 90-130 kDa, about 90-120 kDa, about 90-110 kDa, about 90-100 kDa, about 100-310 kDa, about 100-300 kDa, about 100-290 kDa, about 100-280 kDa, about 100-270 kDa, about 100-260 kDa, about 100-250 kDa, about 100-240 kDa, about 100-230 kDa, about 100-220 kDa, about 100-210 kDa, about 100-200 kDa, about 100-190 kDa, about 100-180 kDa, about 100-170 kDa, about 100-160 kDa, about 100-150 kDa, about 100-140 kDa, about 100-130 kDa, about 100-120 kDa, about 100-110 kDa, about 110-310 kDa, about 110-300 kDa, about 110-290 kDa, about 110-280 kDa, about 110-270 kDa, about 110-260 kDa, about 110-250 kDa, about 110-240 kDa, about 110-230 kDa, about 110-220 kDa, about 110-210 kDa, about 110-200 kDa, about 110-190 kDa, about 110-180 kDa, about 110-170 kDa, about 110-160 kDa, about 110-150 kDa, about 110-140 kDa, about 110-130 kDa, about 110-120 kDa, about 120-310 kDa, about 120-300 kDa, about 120-290 kDa, about 120-280 kDa, about 120-270 kDa, about 120-260 kDa, about 120-250 kDa, about 120-240 kDa, about 120-230 kDa, about 120-220 kDa, about 120-210 kDa, about 120-200 kDa, about 120-190 kDa, about 120-180 kDa, about 120-170 kDa, about 120-160 kDa, about 120-150 kDa, about 120-140 kDa, about 120-130 kDa, about 130-310 kDa, about 130-300 kDa, about 130-290 kDa, about 130-280 kDa, about 130-270 kDa, about 130-260 kDa, about 130-250 kDa, about 130-240 kDa, about 130-230 kDa, about 130-220 kDa, about 130-210 kDa, about 130-200 kDa, about 130-190 kDa, about 130-180 kDa, about 130-170 kDa, about 130-160 kDa, about 130-150 kDa, about 130-140 kDa, about 140-310 kDa, about 140-300 kDa, about 140-290 kDa, about 140-280 kDa, about 140-270 kDa, about 140-260 kDa, about 140-250 kDa, about 140-240 kDa, about 140-230 kDa, about 140-220 kDa, about 140-210 kDa, about 140-200 kDa, about 140-190 kDa, about 140-180 kDa, about 140-170 kDa, about 140-160 kDa, about 140-150 kDa, about 150-310 kDa, about 150-300 kDa, about 150-290 kDa, about 150-280 kDa, about 150-270 kDa, about 150-260 kDa, about 150-250 kDa, about 150-240 kDa, about 150-230 kDa, about 150-220 kDa, about 150-210 kDa, about 150-200 kDa, about 150-190 kDa, about 150-180 kDa, about 150-170 kDa, about 150-160 kDa, about 160-310 kDa, about 160-300 kDa, about 160-290 kDa, about 160-280 kDa, about 160-270 kDa, about 160-260 kDa, about 160-250 kDa, about 160-240 kDa, about 160-230 kDa, about 160-220 kDa, about 160-210 kDa, about 160-200 kDa, about 160-190 kDa, about 160-180 kDa, about 160-170 kDa, about 170-310 kDa, about 170-300 kDa, about 170-290 kDa, about 170-280 kDa, about 170-270 kDa, about 170-260 kDa, about 170-250 kDa, about 170-240 kDa, about 170-230 kDa, about 170-220 kDa, about 170-210 kDa, about 170-200 kDa, about 170-190 kDa, about 170-180 kDa, about 180-310 kDa, about 180-300 kDa, about 180-290 kDa, about 180-280 kDa, about 180-270 kDa, about 180-260 kDa, about 180-250 kDa, about 180-240 kDa, about 180-230 kDa, about 180-220 kDa, about 180-210 kDa, about 180-200 kDa, about 180-190 kDa, about 190-310 kDa, about 190-300 kDa, about 190-290 kDa, about 190-280 kDa, about 190-270 kDa, about 190-260 kDa, about 190-250 kDa, about 190-240 kDa, about 190-230 kDa, about 190-220 kDa, about 190-210 kDa, about 190-200 kDa, about 200-310 kDa, about 200-300 kDa, about 200-290 kDa, about 200-280 kDa, about 200-270 kDa, about 200-260 kDa, about 200-250 kDa, about 200-240 kDa, about 200-230 kDa, about 200-220 kDa, about 200-210 kDa, about 210-310 kDa, about 210-290 kDa, about 210-280 kDa, about 210-270 kDa, about 210-260 kDa, about 210-250 kDa, about 210-240 kDa, about 210-230 kDa, about 210-220 kDa, about 220-310 kDa, about 220-300 kDa, about 220-290 kDa, about 220-280 kDa, about 220-270 kDa, about 220-260 kDa, about 220-250 kDa, about 220-240 kDa, about 220-230 kDa, about 230-310 kDa, about 230-300 kDa, about 230-290 kDa, about 230-280 kDa, about 230-270 kDa, about 230-260 kDa, about 230-250 kDa, about 230-240 kDa, about 240-310 kDa, about 240-300 kDa, about 240-290 kDa, about 240-280 kDa, about 240-270 kDa, about 240-260 kDa, about 240-250 kDa, about 250-310 kDa, about 250-300 kDa, about 250-290 kDa, about 250-280 kDa, about 250-270 kDa, about 250-260 kDa, about 260-310 kDa, about 260-300 kDa, about 260-290 kDa, about 260-280 kDa, about 260-270 kDa, about 270-310 kDa, about 270-300 kDa, about 270-290 kDa, about 270-280 kDa, about 280-310 kDa, about 280-300 kDa, about 280-290 kDa, about 290-310 kDa, about 290-300 kDa, about or 300-310 kDa). In some embodiments, the molecular weight of CH is about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, about 260 kDa, about 270 kDa, about 280 kDa, about 290 kDa, about 300 kDa, or about 310 kDa. In some embodiments, the molar ratio of CH/TPP is about 1:1, about 1.5:1, about 2:1, about 2.5:1, about 3:1, about 3.5:1, about 4:1, about 4.5:1, or about 5:1. Step-2, adding purified CH/TPP NP water suspension dropwise into a covalent crosslinker (ECH, EGDGE, or GA) solution under stirring. In some embodiments, reaction mixture is incubated at about 40° C., about 45° C., about 50° C., about 55° C., or about 60° C. for about 1 hour, about 1.5 hours, about 2 hours, about 2.5 hours, about 3 hours, about 3.5 hours, or about 4 hours. In some embodiments, the final concentration of the reaction mixture for ECH or EGDGE is about 3-7% (e.g., about 3-6%, about 3-5%, about 4-7%, about 4-6%, about 4.5-5.5%, about 3%, about 3.5%, about 4.5%, about 5%, about 5.5%, about 6%, about 6.5% or about 7%) at a pH of about 5-7 (e.g., about 5.5-6.5, about 5, about 6, or about 7). In some embodiments, the final concentration of the reaction mixture of GA is about 0.5-2% (e.g., 0.5-1.5%, about 0.5-1.25%, about 0.75-1.25%, about 0.9-1.1%, or about 0.5, about 0.6, about 0.7, about 0.8, about 0.9, about 1, about 1.1, about 1.2, about 1.3, about 1.4, about 1.5, about 1.6, about 1.7, about 1.8, about 1.9, or about 2) at a pH of about 1-3 (e.g., about 1-2.5, about 1-2, about 1.5-2.5, about 1.75-2.25, about 1, about 1.5, about 2, about 2.5 or about 3). In some embodiments, this step further includes removing unreacted crosslinkers and other impurities by centrifugation.

Step-3, adding purified NP suspension dropwise to a basic (e.g., NaOH) solution under stirring. In some embodiments, the NaOH solution is about 0.001-0.1 M to free the amine sidechain on the CH—NP. The treated CH—NP is purified.

In some embodiments, the association of an anionic radioactive agent (e.g., phosphate P32) with the cationic biopolymer-based NP or MS is performed under acidic condition (e.g., pH of about 4.1, about 4.2, about 4.3, about 4.4, about 4.5, about 4.6, about 4.7, about 4.8, about 4.9 or about 5). In some embodiments, the amount of radioactivity, e.g., phosphate P-32, is about 0.5-20 millicurie (mCi) (equivalent to 1-100 μg).

In some embodiments, the biopolymer hydrogel includes about 11-20% Pluronic™ F127, about 1-10% MC, and about 0.05-1% CH. In some embodiments, the biopolymer hydrogel includes about 11-20%, about 11-19%, about 11-18%, about 11-17%, about 11-16%, about 11-15%, about 11-14%, about 11-13%, about 11-12%, about 12-20%, about 12-19%, about 12-18%, about 12-17%, about 12-16%, about 12-15%, about 12-14%, about 12-13%, about 13-20%, about 13-19%, about 13-18%, about 13-17%, about 13-16%, about 13-15%, about 13-14%, about 14-20%, about 14-19%, about 14-18%, about 14-17%, about 14-16%, about 14-15%, about 15-20%, about 15-19%, about 15-18%, about 15-17%, about 15-16%, about 16-20%, about 16-19%, about 16-18%, about 16-17%, about 17-20%, about 17-19%, about 17-18%, about 18-20%, about 18-19%, or about 19-20% Pluronic™ F127 (w/w). In some embodiments, the biopolymer hydrogel can include about 11%, about 12%, about 13%, about 14%, about 15%, about 16%, about 17%, about 18%, about 19%, or about 20% Pluronic™ F127 (w/w). In some embodiments, the biopolymer hydrogel can include about 1-10% (e.g., about 1-9%, about 1-8%, about 1-7%, about 1-6%, about 1-5%, about 1-4%, about 1-3%, about 1-2%, about 2-10%, about 2-9%, about 2-8%, about 2-7%, about 2-6%, about 2-5%, about 2-4%, about 2-3%, about 3-10%, about 3-9%, about 3-8%, about 3-7%, about 3-6%, about 3-5%, about 3-4%, about 4-10%, about 4-9%, about 4-8%, about 4-7%, about 4-6%, about 4-5%, about 5-10%, about 5-9%, about 5-8%, about 5-7%, about 5-6%, about 6-10%, about 6-9%, about 6-8%, about 6-7%, about 7-10%, about 7-9%, about 7-8%, about 8-10%, about 8-9%, or about 9-10%) MC (w/w). In some embodiments, the biopolymer hydrogel can include about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% MC (w/w). In some embodiments, the biopolymer hydrogel can include about 0.05-1% (e.g., about 0.05-0.95%, about 0.05-0.9%, about 0.05-0.85%, about 0.05-0.8%, about 0.05-0.75%, about 0.05-0.7, about 0.05-0.65%, about 0.05-0.6%, about 0.05-0.55%, about 0.05-0.5%, about 0.05-0.45%, about 0.05-0.4%, about 0.05-0.35%, about 0.05-0.3%, about 0.05-0.25%, about 0.05-0.2%, about 0.05-0.15%, about 0.05-0.1%, about 0.1-1%, about 0.1-0.95%, about 0.1-0.9%, about 0.1-0.85%, about 0.1-0.8%, about 0.1-0.75%, about 0.1-0.7%, about 0.1-0.65%, about 0.1-0.6%, about 0.1-0.55%, about 0.1-0.5%, about 0.1-0.45%, about 0.1-0.4%, about 0.1-0.35%, about 0.1-0.3%, about 0.1-0.25%, about 0.1-0.2%, about 0.1-0.15%, about 0.15-1%, about 0.15-0.95%, about 0.15-0.9%, about 0.15-0.85%, about 0.15-0.8%, about 0.15-0.75%, about 0.15-0.7%, about 0.15-0.65%, about 0.15-0.6%, about 0.15-0.55%, about 0.15-0.5%, about 0.15-0.45%, about 0.15-0.4%, about 0.15-0.35%, about 0.15-0.3%, about 0.15-0.25%, about 0.15-0.2%, about 0.2-1%, about 0.2-0.95%, about 0.2-0.9%, about 0.2-0.85%, about 0.2-0.8%, about 0.2-0.75%, about 0.2-0.7%, about 0.2-0.65%, about 0.2-0.6%, about 0.2-0.55%, about 0.2-0.5%, about 0.2-0.45%, about 0.2-0.4%, about 0.2-0.35%, about 0.2-0.3%, about 0.2-0.25%, about 0.25-1%, about 0.25-0.95%, about 0.25-0.9%, about 0.25-0.85%, about 0.25-0.8%, about 0.25-0.75%, about 0.25-0.7%, about 0.25-0.65%, about 0.25-0.6%, about 0.25-0.55%, about 0.25-0.5%, about 0.25-0.45%, about 0.25-0.4%, about 0.25-0.35%, about 0.25-0.3%, about 0.3-1%, about 0.3-0.95%, about 0.3-0.9%, about 0.35-0.85%, about 0.35-0.8%, about 0.35-0.75%, about 0.35-0.7%, abut 0.35-0.65%, about 0.35-0.6%, about 0.35-0.55%, about 0.35-0.5%, about 0.35-0.45%, about 0.35-0.4%, about 0.4-1%, about 0.4-0.95%, about 0.4-0.9%, about 0.4-0.85%, about 0.4-0.8%, about 0.4-0.75%, about 0.4-0.7%, about 0.4-0.65%, about 0.4-0.6%, about 0.4-0.55%, about 0.4-0.5%, about 0.4-0.45%, about 0.45-1%, about 0.45-0.95%, about 0.45-0.9%, about 0.45-0.85%, about 0.45-0.8%, about 0.45-0.75%, about 0.45-0.7%, about 0.45-0.65%, about 0.45-0.6%, about 0.45-0.55%, about 0.45-0.5%, about 0.5-1%, about 0.5-0.95%, about 0.5-0.9%, about 0.5-0.85%, about 0.5-0.75%, about 0.5-0.7%, about 0.5-0.65%, about 0.5-0.6%, about 0.5-0.55%, about 0.55-1%, about 0.55-0.95%, about 0.55-0.9%, about 0.55-0.85%, about 0.55-0.8%, about 0.55-0.75%, about 0.55-0.7%, about 0.55-0.65%, about 0.55-0.6%, about 0.6-1%, about 0.6-0.95%, about 0.6-0.9%, about 0.6-0.85%, about 0.6-0.8%, about 0.6-0.75%, about 0.6-0.7%, about 0.6-0.65%, about 0.65-1%, about 0.65-0.95%, about 0.65-0.9%, about 0.65-0.85%, about 0.65-0.8%, about 0.65-0.75%, about 0.65-0.7%, about 0.7-1%, about 0.7-0.95%, about 0.7-0.9%, about 0.7-0.85%, about 0.7-0.8%, about 0.7-0.75%, about 0.75-1%, about 0.75-0.95%, about 0.75-0.9%, about 0.75-0.85%, about 0.75-0.8%, about 0.8-1%, about 0.8-0.95%, about 0.8-0.9%, about 0.8-0.85%, about 0.85-1%, about 0.85-0.95%, about 0.85-0.9%, about 0.9-1%, about 0.9-0.95, or about 0.95-1%) CH (w/w). In some embodiments, the biopolymer hydrogel can include about 0.05%, about 0.1%, about 0.15, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% CH (w/w).

### Dosimetry

The dosimetry of the radiotherapeutic hydrogel as described herein (e.g., containing CH—NP/P-32 as micro-brachytherapy) can be calculated based on the retention/kinetic of the radioactivity within the lesion over time post the administration. In a similar manner, the dosimetry can approximately be estimated from the one calculated for the insoluble colloid of CCP P-32 (colloid, without free phosphate P-32), an FDA approved radioactive agent. CCP P-32 has been approved by the FDA for intraperitoneal or intracavitary injection for treatment of peritoneal or pleural effusions caused by metastatic disease. In addition, CCP P-32 has also been approved as interstitial or micro-brachytherapy for intratumoral injection, with a dose based on estimated gram weight of tumor.

Phosphorus P-32 is a pure (100%) beta-emission radioisotope with a physical decay half-life of 14.3 days. The mean energy of the beta particle is 695 keV. Although the maximum range of a P-32 beta particle is 0.8 cm in soft tissue, approximately 50% of the energy can be absorbed in the first 0.1 cm of penetration. An equivalent radiation dose of approximately 7.3 Gy per gram of tumor tissue can be obtained in a concentration of 0.001 mCi/mL or mCi/gram, under an assumption of that P-32 is uniformly distributed with a complete absorption within the lesion. Based on this assumption, a theoretical maximum average radiation dose of approximately 730 Gy can be reached with a radioactive concentration of 0.1 mCi/gram for P-32, used as micro-brachytherapy. Hence, a suggested radioactivity dose of approximately 0.1-0.5 (e.g., about 0.1, about 0.2, about 0.3, about 0.4, or about 0.5) mCi/gram can be used for this disclosure (e.g., as interstitial brachytherapy with an intratumor administration).

A suggested radioactivity dose range is 10 to 20 mCi and 6 to 12 mCi of CCP P-32 for intraperitoneal instillation and intrapleural instillation, respectively. For safety concerns, a careful intracavitary instillation for CCP P-32 is required to avoid placing the dose of radioactivity into intrapleural or intraperitoneal loculations, bowel lumen or into the body wall. Intestinal fibrosis or necrosis and chronic fibrosis of the body wall have been reported to result from unrecognized misplacement of the therapeutic agent. The presence of large tumor masses indicates the need for other forms of treatment. However, when other forms of treatment fail to control the effusion, CCP P-32 can be useful. To obtain an estimate of the average dose, the surface area of the pleural and peritoneal cavities is assumed to amount to 4,000 and 5,000 cm², respectively. The estimated radiation doses to an average patient (70 kg) with 90% retention of a dose of 20 mCi of radioactivity distributed uniformly over these areas has been estimated for CCP P-32. The decreases of the averaged radiation doses at various tissue depths away from the surfaces of the pleural and peritoneal cavities are also tabulated for CCP P-32. Compared to CCP P-32, an even distribution within intrapleural or intraperitoneal cavities can be expected for this disclosure of radiotherapeutic hydrogel.

### Methods of Treatment

The radiotherapeutic hydrogel described in the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject. In some embodiments, the treatment can result in the reduction of the likelihood of recurrence of cancer in a subject.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin.

In one aspect, the disclosure features methods that include administering a therapeutically effective amount of hydrogel disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, or urethral cancer. In some embodiments, the subject has a solid tumor.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the hydrogel is to be administered, a severity of symptoms and a route of administration, and thus administration can be determined on an individual basis.

An effective amount can be administered in one or more administrations. By way of example, an effective amount of the hydrogel is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro.

Effective amounts of radioactivity and schedules for administering the compositions disclosed herein may be determined empirically, and making such determinations is within the skill in the art. A typical one-time usage, biweekly or monthly dosage of an effective amount of the radiotherapeutic hydrogel as described herein (e.g., for peritoneal and thoracic intracavity administration or postsurgical cavity administration) is in an effective dosage range of 1-20 mCi.

In some embodiments, whole body imaging for the radioactivity can be conducted via SPECT or SPECT/CT (bremsstrahlung measurement) over time, i.e., at 0, 1, 2, 4, 8 & 16 days of post injection of the radiotherapeutic hydrogel. The images can be analyzed for the ROIs (region of interest) including e.g., the loading site, liver, spleen, lungs, kidney. The distribution of the radioactivity as % ID (% injected radioactivity) and % ID/g (% injected radioactivity per gram) can be estimated for the ROIs, as well as within the whole body (% ID) over times of post injection.

### Local (e.g., Intratumor or Intracavity) Administration

The administration can be done according to any suitable route of in vivo that is suitable for delivering and “sustained-release” of the therapeutic agents directly into the lesions of the patients. The preferred routes of administration can be apparent to those of skill in the art. Exemplary methods of in vivo administration include, but are not limited to, intraperitoneal administration, intrathoracic administration, intranodal administration, intratumor administration, intramuscular administration, transdermal delivery, intra-postsurgery-cavity direct injection or impregnation via a catheter.

### Intratumoral (IT) Injection

Patients with unresectable, locally advanced, or metastatic solid tumors have a poor prognosis and few therapeutic options, especially after having failed standard care therapies. One of promising adjuvant therapies is direct intratumor (IT) delivery of the therapeutic agents. The intratumor radiotherapy (ITR), also referenced as micro-brachytherapy or interstitial radiotherapy, by directly intratumoral injection of beta-emitting radioactive MS or CCP P-32 has been proven as a relatively safe and effective choice. Conflicting evidence about safety and efficacy might be explained by the considerable variation in the treatment characteristics; hence, a high retention/even distribution within the lesion post administration is critical for its anti-tumor efficacy.

As any other micro-brachytherapy, the application of present disclosure of radiotherapeutic hydrogel can be locally administrated via an intratumoral injection of percutaneous or intraoperative (injection into the unresectable lesions intraoperatively). Percutaneous injection usually needs to be performed under imaging-guidance; challenge in precise injection is expected much less for an intraoperative IT injection. Imaging-guided, such as ultrasound (US) or computerized tomography (CT) or magnetic resonance imaging (MRI), percutaneous core needle biopsy has been used extensively in daily clinical practice for the pathologic confirmation for neoplasms. Imaging-guidance for percutaneous injection is necessary for ITR because inadequate local delivery and inhomogeneous dose distribution result in reduced treatment efficacy and potential complications. As an imaging-guidance tool, US has a number of clear advantages over CT or MRI in core needle biopsy; fewer false-negative biopsies, lack of ionizing radiation, portability, relatively short procedure time, real-time intra-procedural visualization of the biopsy needle, ability to guide the procedure in almost any anatomic plane, and relatively lower cost.

Several factors can impact the distribution and retention of the radioactivity within the lesion, and finally impact the therapeutic efficacy of ITR These factors include gelation time/viscosity of the hydrogel, particle size of NP, administration method, volume and speed of injection, total amount of radioactivity administered, as well as a reliable imaging assay during and after administration. For instance, large size MS can require multiple manual injection locations or in a grid-like pattern, with a small volume in each injection to improve the distribution. Small size NP can be administered through a single infusion technique in the tumor center, assuming that the pressure force could distribute the hydrogel throughout the tumor. Empirically, a grid-like injection procedure with small volume depots is advised over a single large infusion for a large tumor.

For the present disclosure, a needle used for ITR injection could be a needle in size between 18 and 22G (outer diameter 1.2-0.7 mm). An endoscopic ultrasound approach with a needle can be utilized. In addition, the volume and speed of injection can also affect the distribution of hydrogel, and too large volume might result in leakage out of the tumor. With a higher amount of volume, high resistance with a sudden release of syringe pressure is often felt for the leakage during infusion. For this disclosure as ITR, an injection volume of less than 20% of the tumor volume is suggested to ensure an even distribution with minimized possibility of leakage.

Leakage appears to follow the path of least resistance. Potential routes of leakage include external leakage and internal leakage. External leakage from the syringe can occur due to high resistance in the tumor. Internal leakage to surrounding normal tissues can usually be divided in hematogenous or intravenous and intraductal leakage. In the majority of human and animal studies, some degree of intravenous leakage or shunting of particles through the capillary bed has been reported. Some studies reveal a bi-phasic drainage of the injected particles out of the tumor, a fast wash-out phase followed by a slow decline. Lymphatic drainage is an additional potential route. This well-known route of tumor drainage is commonly used in the sentinel node procedure. The MS may presumably be too large for drainage of significant amounts of radioactivity to the draining lymph nodes.

The use of a small needle can reduce the external leakage. However, care should be taken to prevent premature settling and clotting of NP/hydrogel inside the syringe and blocking the needle. A 21G or 18G needle seems to be the preferred size to use. Additional measures to reduce leakage can include slow injection and withdrawal of the needle with slight pressure or injection of obstructing pledged/foam.

In this disclosure, an alternative injection technology can also be used: with a removal of tumor tissue in a reasonable volume by the needle biopsy technology immediately prior to the IT dosing to ensure the administration of required radioactivity/volume with minimized leakage.

A suggested amount of radioactivity for this disclosure as ITR is an average absorbed dose of not less than 100 Gy for the tumor as micro-brachytherapy. The absorbed dose can be calculated based on the retention/kinetical of the radioactivity in the tumor over time. As mentioned previously, a direct IT injection with tumoricidal particles does not automatically lead to an effective tumor treatment. Since an insufficiently absorbed dose, apart from total absorbed dose, an evenly intratumor distribution of the radioactivity is critical, as any missed parts of the tumor can result in residual vital tumors. This disclosure of radiotherapeutic hydrogel can be a potential solution for the challenges of existing ITR, because in addition to its micro-brachytherapy, an extended infiltration of the radioactive agent via “sustained-release” and its selective cytotoxicity to proliferating cancer cells can effectively eradicate the residual tumors surrounding the loading site.

In some embodiments, a volume of 0.2-10 ml of radiotherapeutic hydrogel is administered (e.g., for intratumor administration). In some embodiments, a volume of at least 0.2-5, 1-5, 1-10, 2-10. 5-10 ml of radiotherapeutic hydrogel is administered. In some embodiments, a total radioactivity of 1 to 20 mCi of radiotherapeutic hydrogel is administered. In some embodiments, the exact amount of the radioactivity will be estimated based on the tumor size and dosimetry calculation.

### Peritoneal and Thoracic Intracavity Administration

One of the examples of intracavity injection is peritoneal for ovarian cancer. Ovarian cancer is the most lethal of the gynecologic cancers, with a 5-year survival rate less than 50%. The unique pattern of dissemination as transperitoneal spread is the most common route in ovarian cancer such that, diagnosis, the tumor is confined to the abdominal and pelvic cavity in approximately 85% of patients. Radiation therapy has been historically used in the adjuvant setting for the management of ovarian carcinoma of all tumor subtypes with reasonable results. Because ovarian cancer is rarely confined to the pelvis, whole pelvic radiation is a largely ineffective method of disease control since it does not treat the entire volume at risk of recurrence. However, the low doses required to meet tolerance of the bowel, kidneys, and liver using two dimensional fields are ineffective in eradicating gross residual disease in the peritoneal cavity resulting in poor therapeutic efficacy. Additionally, the toxicity of radiation therapy is particularly high when using wide-field external irradiation. High rates of both acute and late toxicity, particularly gastrointestinal, resulted in the abandonment of radiation in this disease particularly when cisplatin is confirmed to be a highly active systemic agent. Improved radiation techniques with lower toxicity have led to a renewed interest in the use of radiation therapy for metastatic ovarian cancer as well as peritoneal metastases form cancers with other disease origins/sites.

Radioactive insoluble colloids, in particular CCP P-32, have a history of being used as adjuvant treatment for ovarian cancer. With a direct injection into the peritoneal cavity, CCP P-32 has become the agent of choice for the treatment of ovarian cancer and the palliation of malignant ascites since 1960s. Improvement in recurrence rates and even survival has been demonstrated for patients with endometrial cancer and positive peritoneal cytology. However, CCP P-32 for early-stage disease is abandoned due to no difference in survival rate compared to the easier administration and lower toxicity of newer chemotherapeutic agents (platinum). With aggressive therapy at diagnosis, including surgery and platinum-based chemotherapy, more than 80% of women diagnosed with advanced disease can have an initial complete response. Unfortunately, these responses are infrequently durable and the majority of women with ovarian cancer develop recurrent disease, which is typically incurable although subsequent response and months of survival may still be possible. Hence, a new adjuvant treatment with high efficacy and low toxicity is needed to prevent recurrences. This disclosure of radiotherapeutic hydrogel, in the same dosing manner as CCP P-32, with an even distribution within peritoneal/pelvic cavity and selectivity to proliferating cancer cells could be a potential solution. In addition, the formulation of polysaccharide hydrogel can further improve the therapeutic efficacy by the property of preventing peritoneal adhesion.

### Postsurgical Cavity Administration

Another example of treatment (e.g., intracavity administration) is for delivering the localized radiation therapy within a surgical created cavity (e.g., via a catheter or needle). Although treatment modalities vary considerably depending on stage and location, surgical excision is an integral part of treatment for most solid tumors. The goal of surgical resection is the removal of cancer both gross and microscopic. However, incomplete excision as well as residual tumor cells left in the surgical cavity is one of the main reasons of recurrence, and additional post-surgery adjuvant treatments are often prescribed. For example, based on a study of 10 million cancer patients with surgical treatment from year 1998 to 2012 in the largest oncology database, the National Cancer Data Base (NCDB), 6.5 million out of 10 million cancer patients showed surgical margin data for the resected specimen; among them an overall 30% positive surgical margin (PSM) occurs in T4 category of the ten most common solid organ cancers in the USA. The listed ten most common solid cancers are cancers of prostate, breast, lung and bronchus, colon and rectal, urinary bladder, thyroid, kidney and renal pelvis, uterine corpus, oral cavity, ovarian. In one aspect, the disclosure involves treating the subject having these solid cancers with the radiotherapeutic hydrogel as described herein (e.g., after surgical removal or during surgical removal of these solid cancers).

For example, over the last decade, breast-conserving surgery followed by whole breast irradiation became the standard of care for the treatment of early-stage breast carcinoma to prevent the recurrence. However, the necessity of giving whole breast irradiation for all patients after breast conserving surgery has been questioned, due to serious side effects caused by the external beam radiation. In recent years, many studies compared whole breast irradiation with partial breast irradiation, whether in the form of intraoperative single dose radiation or brachytherapy (the treatment of cancer at a short distance with a radioactive isotope placed near, on, or in the lesions or tumor) applied to the surgical site following surgery. Both methods were found to have the same effectiveness while brachytherapy had less toxic side effects.

Another example of importance to prevent the recurrence after the surgical treatment is brain tumor. Current standard of care for brain tumor includes maximal safe surgical resection, followed by a combination of radiation and chemotherapy. Even with advanced treatment, the prognosis of brain tumor is poor, the rate of recurrence is high, with a median survival of 14.6 months. Due to the short mean survival, a 2-year survival of approximately 26%, new therapeutic strategies for brain tumor have been investigated consecutively including local drug delivery approaches. For example, interstitial radiotherapy has been suggested by using I-125 and Ir-192 temporary and permanent implants. Interstitial high-dose-rate therapy needs complicated implantation techniques such as CT-guided surgery, and is frequently associated with relatively high toxicity. Interstitial low-dose-rate therapy, temporary implants are preferred as permanent implants bear an increased risk of prolonged edema. Brachytherapy with temporary implants can lead to prolonged survival in patients with recurrent glioma, but it is associated with morbidity and relatively high costs. A third approach is the GliaSite, a technological alternative to seed-implantation, in which radiation is applied via a surgically inserted balloon catheter which is filled with a liquid I-125 containing solution to deliver a high-dose-rate therapy. This device is showing promising results in the recurrent disease setting; however, this approach has some shortcomings in terms of uncertainties of dose distribution, side effects and invasiveness in a highly palliative treatment setting.

Despite the ability to deliver a high radiation dose, current brachytherapy and/or SIRT cannot fill the surgical cavity which raised questions about the homogeneity of the irradiation on the surgical margins. Therefore, a new approach with an even distribution, less invasive and low toxicity is warranted for almost all locally advanced solid tumors due to frequent recurrences after surgical treatment. In some embodiments, the present disclosure of radiotherapeutic hydrogel including micro-brachytherapy plus “sustained-release” of cytotoxic radioactive agents, can be used to prevent the recurrence. A brief outline of the advantages of the hydrogel or agents being potentially used as a new approach of SIRT is listed as follows: 1). the radiotherapeutic hydrogel can be administered into the surgically created cavity by a catheter or a needle injection and reserves the need for surgical implantation; 2). the radiotherapeutic hydrogel ionizing irradiates the resection margin of the surgical cavity continuously and eliminates the need for repeated visits to receive radiotherapy; 3). the radiotherapeutic hydrogel is biodegradable and avoids the need for surgical removal and component of polysaccharide can also promote wound healing; 4). the “sustained-release” and infiltration of the radiotherapeutic agent within the lesion along with the degradation of the hydrogel can further increase the therapeutic efficacy by selectively killing the proliferating cancer cells with low toxicity to surrounding normal tissues.

### EXAMPLES

The following examples are intended for the purpose of illustration of the present invention. However, the scope of the present invention should be defined as the claims appended hereto, and the following examples should not be constructed as in any way limiting the scope of the present invention.

### Materials

The molecular weight of chitosan (CH) used in the present disclosure is in a range of 10-1,000 KDa with a degree of deacetylation (DD) of greater than 70%. The CH or CH solution can be sterilized by autoclaving (121° C., 20 minutes). A GMP grade of CH, methylcellulose (MC), Pluronic™ F-127, and phosphate P-32 (P-32) can be used for clinical supply. Other chemicals and materials, with high purity and quality, were purchased from vendors and used without any further purification in the following examples.

### Example 1: Preparation of Biopolymer Hydrogels

In this example, the hydrogels were prepared via polysaccharides, e.g., MC and CH, and addition of other polymers and/or crosslinkers, e.g., gelatin, alginate, hyaluronic acid (HA), glutaraldehyde (GA), glycerophosphate (GP), glycerol, tripolyphosphate (TPP), with or without addition of a stabilizer. The formed hydrogels were evaluated and compared on gelation, injectability/viscosity as well as in vitro stability. The gelation time of hydrogels was determined, in a sealed 20-ml vial containing 5 ml hydrogel, under an incubation temperature of 35±2° C. via visual inspection when reversing the container. The viscosity of hydrogels at different temperatures, in a range of 4-37° C., was measured in a rotational viscometer. The in vitro stability of hydrogels was tested by incubation in buffers with different pH, e.g., PBS (pH 7.2) or 0.1 mM acetate buffer (pH 5.5), within a 37° C. water bath.

### Example-1/1: MC/CH/GP Hydrogel Preparation

The hydrogel was prepared by mixing MC, CH, GP and/or glycerol. Briefly, a stock solution of CH (3% w/w) or MC (10% w/w) was dissolved in 0.1 M acetic acid or distilled water, respectively, and then kept in refrigerator overnight to ensure complete dissolution. To a certain volume, e.g., 10 ml, of the cooled and diluted CH solution under magnetic stirring, a different predetermined volume of the chilled 10% MC and 45% (w/w) GP aqueous solution with or without glycerol were added dropwise to obtain clear and homogeneous hydrogel solutions, which were kept under continuously mixing for an additional 10 minutes at room temperature. The pH of the final hydrogel solutions was adjusted to approximately 6±1. A gelation time of approximate 3 minutes under 37° C. incubation was obtained with an optimized formulation, e.g., 5% MC/0.1% CH/3.6% GP (w/w).

### Example-1/2: CH/Crosslinker Hydrogel Preparation

In another embodiment, the hydrogel was prepared by adding and mixing CH solution with crosslinkers, e.g., gelatin, alginate, HA, glutaraldehyde, and/or TPP. Briefly, a stock solution of CH (3% w/w) was prepared in 0.1 M acetic acid, and stock solutions of crosslinkers (10% w/w) were prepared in distilled water. All stock solutions were prepared at room temperature and then kept in refrigerator overnight to ensure a complete dissolution. To a certain volume, e.g., 10 ml, of the cooled CH solution under magnetic stirring, a different predetermined volume of the chilled crosslinker aqueous solution was added dropwise to obtain clear and homogeneous hydrogel, which was kept under continuously mixing for an additional 10 minutes at room temperature to ensure a complete mixing. The pH of the final hydrogel solutions was adjusted to approximately 6±1. A gelation time of approximately 5 minutes under 37° C. incubation could commonly be obtained while keeping a relative low concentration of crosslinkers. However, the viscosity of the hydrogel was significantly increased with a high (>1 mg/ml) concentration of the crosslinker. The relatively high viscosity of hydrogels formed in this manner could be properly used for implantation.

### Example-1/3: Thermoreversibly Sensitive and Injectable Hydrogel Preparation

This example specifically described a procedure for the thermoreversibly sensitive and injectable hydrogel, which was a combination of Pluronic™ F127, MC and CH with or without additional stabilizers. Briefly, stock solutions of Pluronic™ F127 and MC were prepared in distilled water with a concentration of 30% and 10% (w/w), respectively. A stock solution of CH (3% w/w) was prepared in 0.1 M acetic acid. All stock solutions were prepared at room temperature and then kept in a 4° C. refrigerator overnight to ensure a complete dissolution. The hydrogels were prepared in combining with different final concentration ranges of Pluronic™ F127/CM/CH, and their gelation and strength or in vitro stability were evaluated and compared. For example, a range in final concentration of 11-20/a (w/w), 2.0-5.0% (w/w), and 0.2-1.0% (w/w) were assessed for Pluronic™ F127, MC, and CH, respectively. The final pH of the hydrogels was adjusted, and a pH of either 5 or 7 was selected to test the impact of pH on the gelation. The gelation time of hydrogel, in a sealed 20-ml vial containing 5 ml sample, was measured by incubating the vial in a 36±1° C. water bath via visual inspection. The viscosity of hydrogels formulated with different parameters listed above were measured in a 100 ml vail containing 50 ml sample solution. In addition, the impact of a trace amount of crosslinker, such as TPP or ammonium sulfate, on the formation of the hydrogel was also assessed. Although the addition of MC and/or CH could significantly lower the critical micelle temperature (CMT) or critical micelle concentration (CMC), the results demonstrated that a minimal concentration of 12% (w/w) of Pluronic™ F127 was required for gelation under an incubation temperature of 35±2° C. More specifically, a proper gelation time of approximately 2 minutes and injectability could be obtained under an optimized formulation of hydrogel, e.g., in a final concentration of 13%, 4% and 0.2-0.5% (w/w) for Pluronic™ F127, MC and CH, respectively.

In addition to the biopolymer-based hydrogels prepared above, the addition of a stabilizer or radiolysis protecting agents (e.g., ascorbic acid, methionine, or gentisic acid) was assessed, and results demonstrated that there was no significant impact on the gelation and strength of the hydrogels by adding the stabilizer with a final concentration of no greater than 0.2% (w/w).

### Example 2: Synthesis of CH-Based Nanoparticles (CH—NP)

This example has been conducted to evaluate the impact of different parameters, such as reaction pH, concentration of and molecular weight of CH, different crosslinker and different molar ratio of CH:crosslinker as well as with or without addition of stabilizer, on size and strength of the formed NPs. Two different molecular weights (small MW 50-190 kDa, and medium MW 190-310 kDa, with a DD of about 92%) of CH were used in this example for comparison. At a low pH, the amine sidechain on CH-based NP becomes cationic which can act as a natural binder to retain anions (e.g., phosphate), which is essentially the basis for ion-exchange chromatography. The CH-based NPs can vary in effectivity and affinity for complexation of anionic radioactive agents based on the content of amino functional groups. Hence, besides its size and stability, the density of amine or zeta potential of the CH-based NPs is also critical.

### Example-2/1: CH-Based NP Formed in High pH Suspension

CH is a weak base and insoluble in water or organic solvent. However, it is soluble in dilute acidic aqueous solutions. For instance, a CH solution is usually prepared by dissolving CH in 0.1 M acetic acid or HCl. This example described the process of creating CH—NP by suspension in 0.1 M NaOH. Briefly, a low concentration of CH solution (2 or 5 mg/ml) was prepared by diluting the CH (3% w/w) stock solution in distilled water. The formation of CH—NP was conducted in a 50-ml glass vial. To 30 ml of 0.1 M NaOH solution under magnetic stirring, 20 ml CH solution in 2 or 5 mg/ml, in a 20-ml syringe equipped with a 12G needle, was added dropwise. The reaction mixture was kept stirring at ambient temperature for another hour. The formed CH—NP was separated and washed (resuspended via pipetting) in distilled water by centrifugation at 2500 rpm for 25 minutes in a Beckman Allegra X-12 centrifuge. The results demonstrated that the size of NP would be slightly impacted by the start concentration and its molecular weight of CH. However, the CH—NP formed in this manner was not stable, and would be re-dissolved in the suspension of 0.5 mM acetic acid (pH of about 4.0) within approximately 20 minutes.

### Example-2/2: Synthesis of CH-Based NP with Small Molecular Ionic Crosslinkers

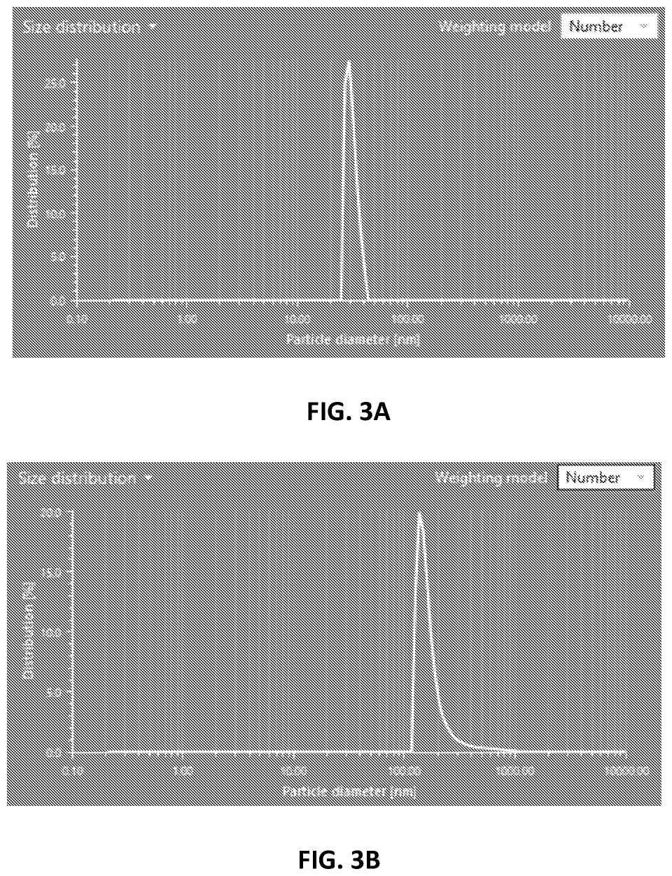

This example described the process of creating CH-based NP with small molecular ionic crosslinkers, e.g., citrate or TPP. Briefly, a stock solution of CH (3% w/w) was prepared by dissolving the CH in 0.1M acetic acid, and then kept in a 4° C. refrigerator overnight to ensure a complete dissolution. A stock solution (5% w/w) of citrate or TPP was prepared in distilled water. CH solutions in a low concentration of (0.5, 1, 2, 5 or 10 mg/ml) were prepared by diluting the CH stock solution (3% w/w), ensuing approximately 5% molar excess of acetic acid as compared to glucosamine in CH. A range of reaction pH tested in this example was between 3-6, which was adjusted by 0.1 M acetic acid. A proper concentration of crosslinker, e.g., citrate or TPP, was prepared in order to keep a constant volume ratio of CH to crosslinker of 2:1 for the reaction. The synthesis of CH-based NP was conducted in a 20-ml glass vial, with a start CH concentration of 0.5, 1, 1.5, 2, 3, or 5 mg/ml. To 10 ml CH solution under magnetic stirring, 5 ml of crosslinker solution in a 5-ml syringe equipped with a 12G needle was added dropwise. The reaction mixture was kept stirring at ambient temperature for another hour or overnight (e.g., more than 8 hours). The formed CH-based NP was separated and washed in distilled water by either centrifuging or dialyzing. For example, the formed NP was pelleted and water washed (resuspended via pipetting) by centrifugation at 2500 rpm for 25 minutes in a Beckman Allegra X-12 centrifuge. The results demonstrated that the size and zeta potential of CH-based NP were significantly impacted by the pH, starting concentration and MW of CH, and molar ratio of CH/crosslinker. For example, FIGS. 3A-3B demonstrates the impact of CH:TPP ratio on the size of CH/TPP NP. In this experiment, the CH/TPP NP was created with a final CH concentration of 1 mg/ml under a reaction pH of approximately 4. An average NP size of 30 nm or 170 nm was observed with a CH:TPP ratio (w/w) of 3:1 or 2.5:1, respectively (FIGS. 3A-3B). In addition, the CH-based NP synthesized with crosslinker TPP (CH/TPP) was stable in both water and 0.5 mM acetic acid (pH of about 4.0) suspensions over a few weeks' storage at ambient temperature. By contrast, the CH-based NP created with citrate crosslinker was unstable and CH/citrate NP was dissolved in 0.5 mM acetic acid (pH of about 4.0) suspension within 20 minutes. Moreover, no significant impact on the size of CH/TPP NP was noticed with an addition of surfactant, e.g., 0.02% Tween 80 or Pluronic™ F127, in the reaction mixture.

### Example-2/3: Synthesis of CH-Based NPs with Anionic Polymers

This example described the process of creating CH-based NP crosslinked with anionic polymers, e.g., alginate, kappa-carrageenan, carboxymethylcellulose, hyaluronic acid, and/or polyglutamic acid. Briefly, a stock solution of CH (3% w/w) was prepared by dissolving the CH in 0.1 M acetic acid, and then kept in a 4° C. refrigerator overnight to ensure a complete dissolution. The stock solutions of anionic biopolymers (1.5% w/w) were first dissolved in distilled water in a hot-water bath (about 50° C.) and then left to cool to room temperature. The reaction solution of CH and anionic biopolymers were prepared by diluting the stock solutions with distilled water to a final concentration of 2 or 1 mg/mL, respectively. The synthesis of NP was conducted in a 20-ml glass vial. To 10 ml CH solution under magnetic stirring, 5 ml of crosslinker solution in a 5-ml syringe equipped with a 12G needle was added dropwise. The results demonstrated a challenge in size controlling for the NPs formulated in this manner.

### Example 3: Synthesis of CH-Based NP with Covalent Crosslinkers

This example described a process of CH-based NP synthesis with covalent crosslinkers, e.g., Epichlorohydrin (ECH), Ethylene glycol diglycidyl ether (EGDGE), and Glutaraldehyde (GA). Because the density of amine on the CH-based NP is critical for an effective complexation of anionic radioactive agents in this disclosure, the reactions described here were crosslinking the primary hydroxyl group inside CH, while its primary amine groups being protected by TPP. Briefly, there were three steps in the synthesis of CH-based NP. Step-1: CH/TPP NP formation. The CH/TPP NP was synthesized and purified as described in Example 2/2. Step-2: crosslinking with primary hydroxyl group of CH. The purified CH/TPP NP water suspension was added dropwise into ECH, EGDGE or GA solution under stirring and incubated at approximately 50° C. for 2 hours. The final concentration in the reaction mixture for ECH, EGDGE, and GA was 5%, 5% and 1%, respectively. The pH of the reaction mixture was approximately 6 “as is” for ECH and EGDGE. The pH of the reaction mixture was adjusted by 0.1 M HCl to approximately 2 for GA. Step-3: TPP removal. The CH/TPP/crosslinker NP synthesized in Step-2 was purified and washed by water (resuspended via pipetting, twice) to remove unreacted crosslinkers and other impurities by centrifuging at 2500 rpm for 25 minutes in a Beckman Allegra X-12 centrifuge. The purified CH/TPP/crosslinker NP suspension was added dropwise into 0.1 M NaOH under stirring. After stirring and incubating at room temperatures for 1 hour, the CH-based NP was purified and washed by water (resuspended via pipetting, twice) to remove the released TPP and other impurities by centrifuging at 2500 rpm for 25 minutes in a Beckman Allegra X-12 centrifuge. The size and zeta potential of the CH-based NPs were measured. Their stability in 0.5 mM acetic acid (pH of about 4.0) suspension stored at room temperature over time was also assessed. Aggregation of CH-based NPs in Step-3 was observed for crosslinker EGDGE and GA. No significant change in NP size was observed for ECH during the Step-3.

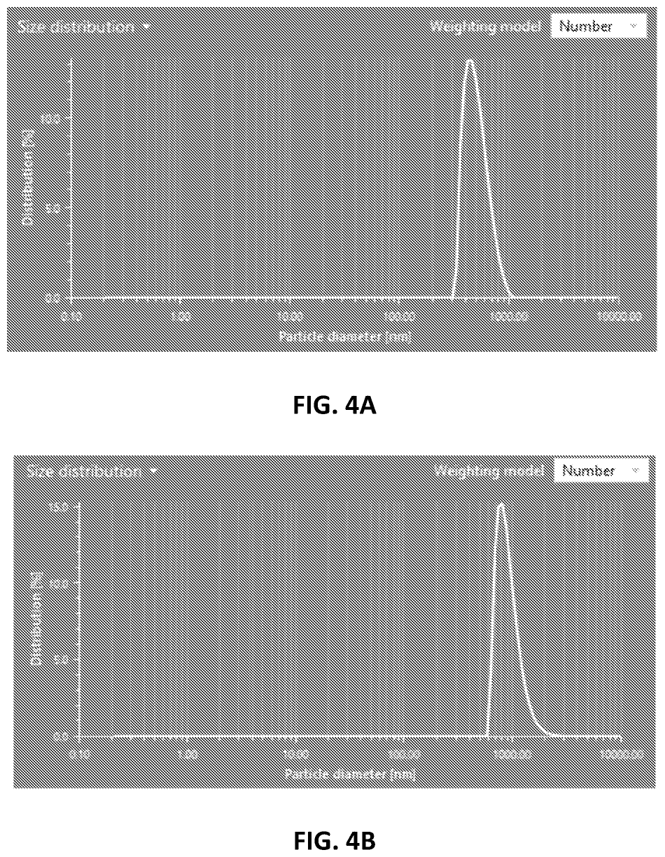

As an example, FIGS. 4A-4B demonstrates the impact of difference in CH molecular weight on the size of CH-based NPs synthesized in this manner. In this experiment, the CH/TPP NP (Step-1) was synthesized with a final CH concentration of 1 mg/ml, a CH/TPP ratio of 2:1 (w/w), and under a reaction pH of approximately 4. An average NP size of 512 nm or 927 nm was observed with a CH molecular weight of 50-190 kDa (small MW, top) or 190-310 kDa (medium MW, bottom), respectively (FIGS. 4A-4B). The size and surface charge of the NP can be further revised by varying the parameters as listed above, if necessary.

### Example 4: Biopolymer-Hydrogel Characterization

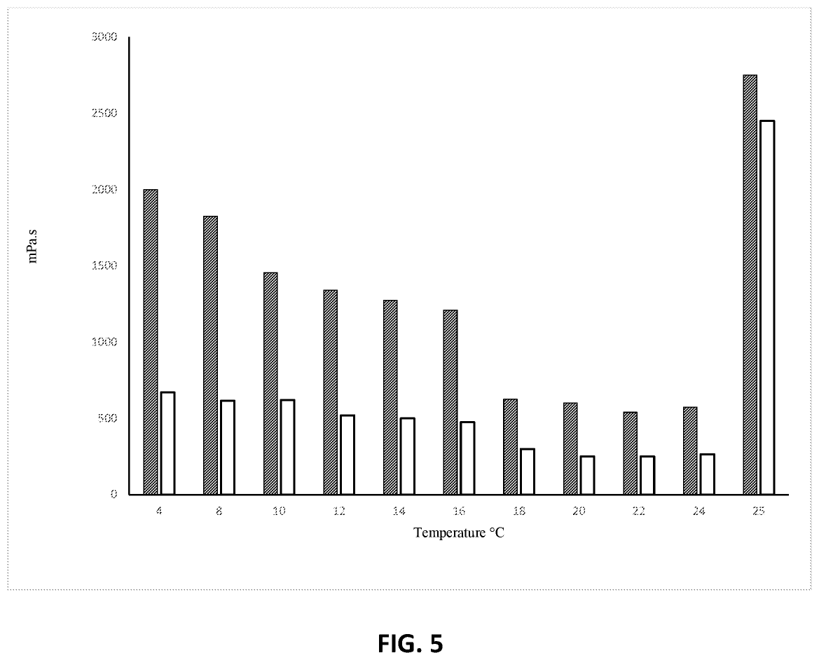

More particularly the following example described the characterizing of thermoreversibly sensitive hydrogels formulated as described in Example 1/3. The characters of hydrogels assessed here include gelation, viscosity, and in vitro stability incubated in buffers with different pH at 37° C. Pluronic™ F127, a nonionic surfactant, exhibits gelation based on micellization above the critical micelle temperature (CMT) or critical micelle concentration (CMC) in water. The presence of biopolymers, e.g., MC and CH, might cause a polymer-assisted interconnected network of micelles, lowering CMT of Pluronic™ F127 and increasing the strength of the hydrogel. The mixture of Pluronic™ F127, MC and/or CH exhibited thermoreversibly sensitive gelation upon heating, and the strength of the formed gel depended on the concentration and temperature. Based on conditions evaluated in Example-1/3, hydrogel with 13% F127/4% MC (w/w) was selected for characterization. The gelation and viscosity versus temperature of hydrogel 13% F127/4% MC with or without additional 0.5% CH were further assessed and compared (FIG. 5). The in vitro stability of hydrogels was tested in a sealed 7-ml vial containing 2 ml of sample. After gelation of the hydrogel by incubation within a 37° C. water bath for approximately 3 minutes, 5 ml prewarmed buffer, either PBS (pH 7.2) or 0.1 mM acetate buffer (pH 5.5), was added. The volume of gel was checked via visual inspection by reversing the vial at 0, 1, 3, 7, 10, 14 and 21-days post incubation. The results demonstrated that the addition of 0.5% CH did not significantly change the manner of gelation although increased the viscosity. FIG. 5 showed that the viscosity of the hydrogels was the lowest at room temperature (18-24° C.), slightly increased under storage in 4° C., and gelation started overtime when the temperature reached 25° C.

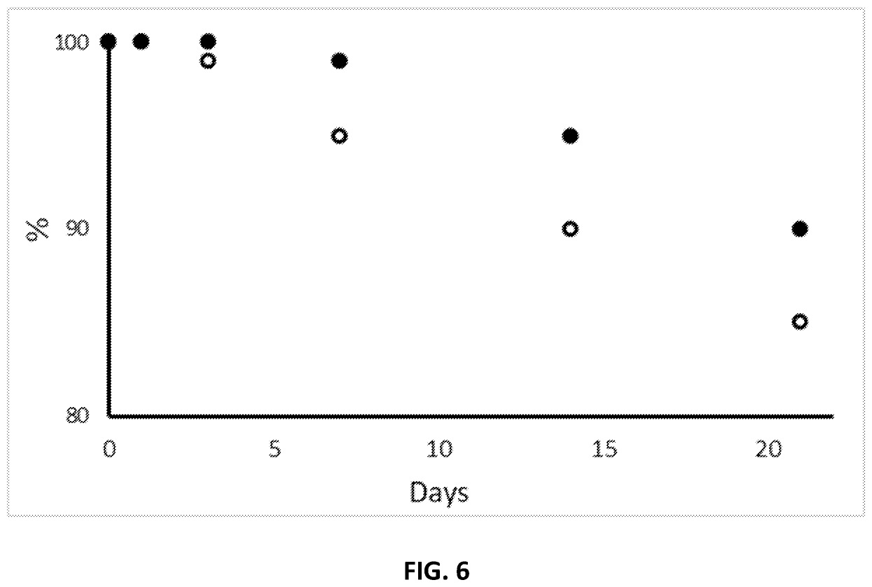

The results demonstrated the hydrogel of 13% F127/4% MC (w/w) was stable, and the stability could be further increased by adding additional 0.5% CH (w/w). FIG. 6 shows the in vitro stability of the hydrogel of 13% F127/4% MC/0.5% CH evaluated and compared in buffers in different pH, e.g., PBS (pH 7.2) versus 0.1 M citrate buffer (pH 5.5), under 37° C. over 3 weeks. The results demonstrated that more than 80% of hydrogel was remained after 3 weeks incubation at 37° C. In addition, the hydrogel was more stable at neutral PBS as compared to that in acidic citrate buffer (FIG. 6).

### Example 5: Association of Phosphate P-32 with CH-Based NP

This example described the association of phosphate P-32 (P-32) on CH-based NP synthesized as discussed in Example 3. In this example, certain amount, e.g., 15 mg, of CH-based NP (CH—NP) was used for evaluation of the impact of pH, amount of P-32 as well as CH—NP synthesized with different crosslinkers. At acidic reaction conditions, the amine sidechain on CH-based NPs becomes cationic which can act as a natural binder to retain anions (e.g., phosphate), which is essentially the basis for ion-exchange chromatography. The yield of association of the CH—NP/phosphate P-32 (CH—NP/P-32) could be evaluated by assays such as instant thin layer chromatography (iTLC) or spectrophotometric assay. The iTLC analysis could be performed with a developing solution of MeOH:H₂O:acetic acid (49:49:2). Rf value for free phosphate P-32 and CH—NP/P-32 would be determined. Currently, P-32 in both Phosphatic Acid P-32 (H₃³²PO₄, with a specific activity of approximately 290 Ci/mg) and Sodium Phosphate P-32 are commercially available. Hence, both forms of P-32 were tested in this example. Spectrophotometric assays were used for the analysis for mimic reactions of CH—NP/P-32, where non-radioactive phosphate was tested in the place of P-32. In spectrophotometric assays, the CH—NP/P-32 reaction solution was centrifuged, the amount of phosphate in the supernatant was measured and compared to the total amount of phosphate added. Detailed information of the assay can be found in the reference of Method 365.3: Phosphorous, all forms, United States Environmental Protection Agency. The results demonstrated that high association yield (%) of P-32 could be obtained under acidic conditions (pH 4-5) with a stability of CH—NP/P-32 over one week storage in water suspension (Tables 1 and 2). In addition, the data showed no significant impact of the amount of P-32, within a tested range of 22-98 μg phosphate, on the association yield (%) and stability of CH—NP/P32 with an approximately amount of 15 mg CH—NP.

TABLE 1
The impact of pH on the binding yield (%) and stability^{‡} of
NaH₂PO₄ to CH—NP, (mean ± SD; n = 3).

| Amount of | | | |
|---|---|---|---|
| NaH₂PO₄^{†} | pH~6.0 | pH 4~4.5 | pH 4~4.5^{‡} |
| 22.0 μg | 58.8 ± 11.9* | 98.5 ± 0.8* | 99.2 ± 1.1 |
| 43.9 μg | 51.2 ± 10.4* | 97.0 ± 0.8* | 97.4 ± 2.0 |
| 87.9 μg | 55.8 ± 6.1** | 95.0 ± 1.2** | 96.3 ± 2.9 |

^{†}A range of 22.0-87.9 μg NaH₂PO₄ incubated with 4 ml CH—NP (15 mg) suspension at room temperature for 60 min. The suspension was centrifuged and amount of the phosphate in the supernatant was measured.
^{‡}Stability of CH—NP bounded phosphate was assessed via keeping it in water suspension at ambient temperature for one week.
*0.05 > p > 0.01;
**p < 0.01

TABLE 2
The impact of pH on binding yield (%) and stability^{‡} of
H₃PO₄ to CH—NP (mean ± SD; n = 3).

| Amount of H₃PO₄^{†} | pH~6.0 | pH 4~4.5 | pH 4~4.5^{‡} |
|---|---|---|---|
| 24.5 μg | 72.7 ± 12.2* | 97.7 ± 1.1* | 98.3 ± 1.7 |
| 49.0 μg | 73.2 ± 7.7* | 96.4 ± 1.1* | 97.1 ± 0.2 |
| 98.0 μg | 77.3 ± 2.2** | 95.4 ± 0.6** | 94.8 ± 0.8 |

^{†}A range of 24.5-98.0 μg H₃PO₄ incubated with 4 ml CH—NP (15 mg) suspension at room temperature for 60 min. The suspension was centrifuged and amount of the phosphoric acid in the supernatant was measured.
^{‡}Stability of CH—NP bound phosphoric acid was assessed via keeping it in water suspension at ambient temperature for one week.
*0.05 > p > 0.01;
**p < 0.01

In addition, the kinetic of association was also assessed and the results demonstrated that a complete association could be obtained within 20 minutes incubation at room temperature (Table 3).

TABLE 3
The impact of time on binding yield (%) of PO₄³⁻ to
CH—NP (mean ± SD; n = 4).

| | 10 min | 20 min | 40 min | 60 min |
|---|---|---|---|---|
| Bound (%) | 94.0 ± 2.7* | 95.3 ± 2.8 | 95.3 ± 2.6 | 95.4 ± 2.2 |

10 μg phosphorus (equivalent to an approximate 44 μg and 32 μg of NaH₂PO₄ and H₃PO₄, respectively) incubated with 4 ml CH—NP (15 mg) suspension at room temperature for over time. The suspension was centrifuged and amount of the phosphoric acid in the supernatant was measured.
*0.05 > p > 0.01

### Example 6: In Vivo “Sustained Release” Assessment

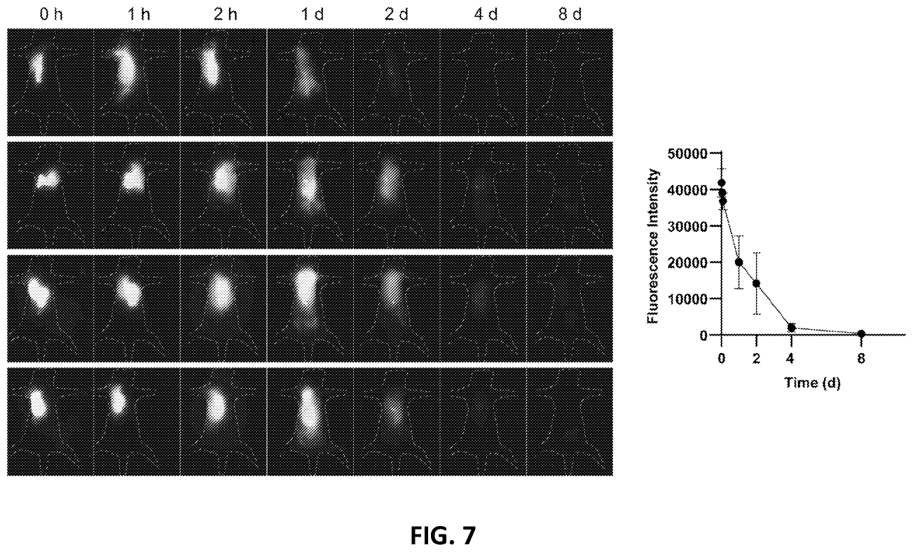

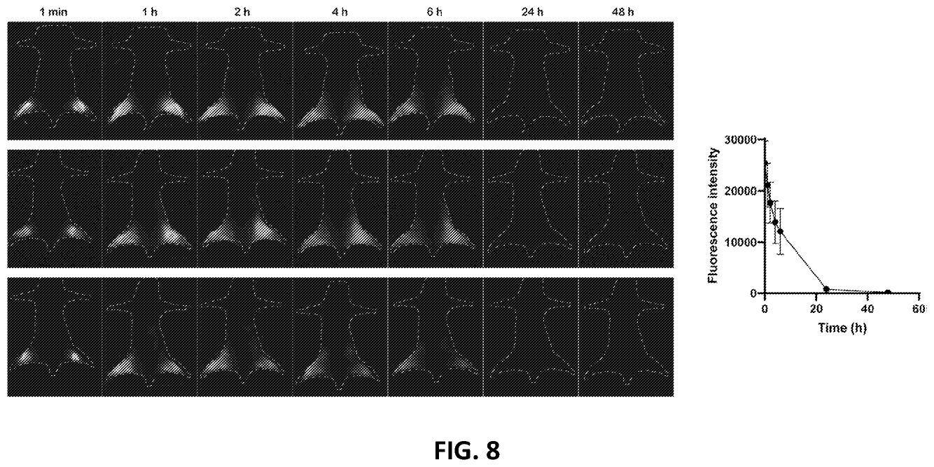

This example described the in vivo “sustained release” of small molecule from hydrogel in healthy Balb/C mouse via optical imaging. The hydrogel tested in this animal experiment was prepared as 13% F-127/4% MC (w/w), which containing 0.2 mg/ml fluorescent indocyanine green (ICG). The hydrogel was prepared, kept in a 4° C. refrigerator overnight ensuring a complete mixing, and balanced to ambient temperature prior to the administration. The molecular weight of ICG is 775 Da, which was used here as an example of small molecule. Two animal groups with different route of subcutaneous (s.c.) or intramuscular (i.m.) injection was assessed with an injection volume of 500 μl or 50 μl, respectively. The imaging was conducted for the animals immediately after hydrogel administration (within 1 minute), and then at predetermined time points of post injection. The results demonstrated a relatively fast rate of “sustained release” of TCG with a clearance half-life of approximately 24 or 4 hours for s.c. (500 μl; FIG. 7) or i.m. (50 μl; FIG. 8), respectively. Moreover, an initial diffusion of hydrogel from the loading site to surround areas was observed within first 5 minutes post s.c. injection. However, no significant difference in volume of hydrogel was noticed (vial inspection) during the 8-day study period (the animal group with 500 μl s.c. administration).

### Example 7: In Vitro Assessment of “Sustained Release” of P-32 from Hydrogel

This example can be used to assess the “sustained release” rate of P-32 from radioactive hydrogel in in vitro incubation (37° C.) over time. The radioactive hydrogel can be prepared as described in Example 3 which can be dispensed and immobilized with CH—NP/P-32 as described in Example 4. The in vitro “sustained release” of P-32 from hydrogels can be determined in a sealed 7-ml vial containing 2 ml of sample. After gelation of the hydrogel by incubation in a 37° C. water bath for approximately 3 minutes, 5 ml prewarmed buffer, either PBS (pH 7.2) or 0.1 mM acetate buffer (pH 5.5), can be added on the top of the hydrogel. At predetermined time intervals, e.g., 0.083, 0.5, 1, 2, 4, 8 hours, and 1, 3, 7, 10, 14 and 21 days post-incubation, an aliquot of 20 μl of the supernatant can be collected and their radioactivity can be measured. The rate (%) of “sustained release” can be calculated as the measured radioactivity in supernatant out of the total radioactivity added, with the radioactivity decay correction. The impact of pH on the rate of “sustained release” can be assessed by comparing the rate of “sustained release” in buffers in different pH, e.g., PBS (pH 7.2) vs 0.1 mM acetate buffer (pH 5.5). Moreover, if necessary, the components, e.g., the final concentrations of F-127/CM/CH in the radioactive hydrogel can be further optimized based on the rate of “sustained release” obtained in this experiment accordingly.

### Example 8: Radioactivity Retention, Tissue Distribution, Kinetic and Dosimetry of the Radiotherapeutic Hydrogel in Healthy Mice

This example can assess the radioactivity retention in the loading site, tissue distribution, and kinetics for the radiotherapeutic hydrogel in healthy mice. The radiotherapeutic hydrogel can be prepared as described in Example 3, which can be dispensed and immobilized with CH—NP/P-32 as described in Example 4. Two different routes of subcutaneous (s.c.) and intramuscular (i.m.) administration can be assessed, with an injection volume of 0.2 ml in approximately 0.1 mCi per mouse (2 groups: n=5; total=10 mice). Whole body imaging for the radioactivity can be conducted via SPECT or SPECT/CT (bremsstrahlung measurement) over time, i.e., at 0, 1, 2, 4, 8 & 16 days of post injection of the radiotherapeutic hydrogel. The images can be analyzed for the ROIs (region of interest) including the loading site, liver, spleen, lungs, kidney. The distribution of the radioactivity as % ID (% injected radioactivity) and % ID/g (% injected radioactivity per gram) can be estimated for the ROIs, as well as within the whole body (% ID) over times of post injection. In addition, after the last imaging, the animal can be terminated, tissues of interest, e.g., lungs, heart, liver, spleen, stomach, small and large intestines, kidneys, skin, muscle, bone and blood can be dissected, collected and weighted. Their radioactivity can be measured. The data can be calculated and reported as % ID and/or % ID/gram. Based on imaging data, radioactivity distribution in tissues of interest over time and the dosimetry can be calculated accordingly.

### Example 9: Preclinical Efficacy Assessment

This example describes the preclinical efficacy assessment of the radiotherapeutic hydrogel in B16 marine melanoma model in C57BL/6 mice. The melanoma selected for this assessment is characterized as a radiation-resistant tumor. The radiotherapeutic hydrogel can be prepared as described in Example 3, which can be dispensed and immobilized with CH—NP/P-32 as described in Example 4. Briefly, when the tumor reaches approximately 300 mm³, a volume of 100 μl of radiotherapeutic hydrogel with approximately 10 or 50 μCi radioactivity can be administered via an intratumor injection. The therapeutic efficacy can be assessed by measuring the tumor size and/or survive rate over time after a single treatment of the radiotherapeutic hydrogel. The hydrogel alone without radioactivity as well as sodium phosphate P-32 solution (100 μl with 50 μCi of radioactivity) can be used as controls (total 4 groups with two therapeutic groups and two control groups; n=10, total=40 mice). If needed, the imaging can be conducted at, 1, 2, 4, 8 & 16 days post injection by SPECT/CT (bremsstrahlung) or optical imaging via Cerenkov luminescence imaging.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Alimi K. A., Firusin N. & Dempke W Effects of intralesional 32P chromic phosphate in refractory patients with head and neck tumors. Anticancer Res 2007; 27(4):2997-3000
Bakker R. et al., Intratumoral treatment with radioactive beta-emitting microparticles: a systemic review. J Radiat Oncol 2017; 6:323
Berlin N. I. Treatment of the myeloproliferative disorders with 32P. Eur J Haematol 2000:65:1-7.
Bult W, Vente M. A. D. D, Vandermeulen E, Gielen I, Seevinck P. R., Saunders J. et al. Microbrachytherapy using holmium-166 acetylacetonate MSs: a pilot study in a spontaneous cancer animal model. Brachytherapy United States 2013; 12(2):171-177
Bussu F, Tagliaferri L, Mattiucci G, Parrilla C, Dinapoli N, Micciche F. et al. Comparison of interstitial brachytherapy and surgery as primary treatments for nasal vestibule carcinomas. Laryngoscope 2016; 126(2):367-371
Calixto G., Annunzin S. R., Victorelli F. D. et al. CH-based drug delivery systems for optimization of photodynamic therapy: a review. AAPS Pharm Sci Tech 2019; 20:253
Cheng Y., Kiess A. P., Herman J. M. et al. Phosphoruse-32, a clinically available drug, inhibits cancer growth by inducing DNA double-strand breakage. PLOS One 2015; 0128152
Fields E. C., McGuire W. R., Lin L. & Temkin S. M. Radiation treatment in women with ovarian cancer: past, present, and future. Frontiers in Oncology 2017; 7:177-190.
Goh A., Chung A, Lo R H G, Lau T. N., Yu S., Chng M. et al. A novel approach to brachytherapy in hepatocellular carcinoma using a phosphorus 32 (32P) brachytherapy delivery device-a first-in-man study. Int J Radiat Oncol Biol Phys 2007; 67(3):786-792
Harrison C., Bareford D., Butt N. et al. Guidelines for the investigation and management of adults and children presenting with thrombocytosis. Brit J Haematol. 2010; 149:352-375
Huang F., Gan G. Y, Lin W. Y et al. Investigation of the local delivery of an intelligent CH-based 188Re thermosensitive in situ-forming hydrogel in an orthotopic hepatoma-bearing rat model. J Radiational Nucl Chem 2014; 299:31-40
Jozwiak T, Filipkowska U., Szymczyk P. & Mielcarek A. Sorption of nutrients (orthophosphate, nitrte III and V) in an equimolar mixture of P—PO₄, N—NO2 and N—NO3 using CH. Arabian J Chem 2016; 4:8.
Kean T. & Thanou M. Advanced drug delivery review. Adv Drug Del Rev 2010; 62:3-11.
Kim J. K., Han K-H., Lee J. T. et al: Long-term clinical outcome of phase Ub clinical trial of percutaneous injection with Holmium-166/CH complex (Milican) for the treatment of small hepatocellular carcinoma. Clin Cancer Res 2006:12(2):503-8
Lawrence J. H. Nuclear physics and therapy: Preliminary report of a new method for the treatment of leukemia and polycythemia. Radiology. 1940; 52:51-60.
McMullin M. F. et al. Guidelines for the diagnosis, investigation and management of polycythemia/erythrocytosis. Brit J Haematol. 2005; 130:174-195.
Qu X. & Weinberger J. Encapsulation of isotope on novel b-emitting poly(ethylene terephalate) surface. J Biomed Mater Res. 2001; 57:619-623.
Rosemurgy A., Luzardo G., Cooper J., Bowers C., Zervos E., Bloomston M. et al. 32P as an adjunct to standard therapy for locally advanced unresectable pancreatic cancer: a randomized trial. J Gastrointest Surg 2008; 12(4):682-688
Schnurch A. B & Dunnhaupt S. CH-based drug delivery system. Eur J Pharm Biopharm 2012; 81:463-9
Tennvall J. & Brans B. EANM procedure guideline for 32P phosphate treatment of myeloproliferative diseases. Eur J Nucl Med Mol Imaging 2007; 34:1324-7
Tong A., Kao Y. H., Too C. W. et al. Yittrium-90 hepatic radioembolization: clinical review and current techniques in interventional radiology and personalized dosimetry. Br J Radiol 2016; 89:20150943
Vimalnath K. V, Shetty P., Shakraborth S. et al. Practically of production of 32P by direct neutron activation for its utilization in bone pain palliation as Na3[32P]P04. Cancer Biotherapy Radiopharm 2013; 28:423-28
Zhu J. L., Yu S. W. K., Chow P. K. H. et al. Controlling injectability and in vivo stability of thermogelling copolymers for delivery of yttrium-90 through intra-tumoral injection for potential brachytherapy. Biomaterials 2018; 180:163-72.

What is claimed is:

1. A radiotherapeutic hydrogel, comprising:
a) a biopolymer hydrogel;
b) an anionic radioactive agent; and
c) a cationic biopolymer or a cationic biopolymer-based nanoparticles (NP) or microspheres (MS);
wherein the cationic biopolymer or the cationic biopolymer-based NP or MS associate with the anionic radioactive agent and are dispersed and/or immobilized in the hydrogel.

2. The radiotherapeutic hydrogel of claim 1, wherein the biopolymer hydrogel is injectable or implantable.

3. The radiotherapeutic hydrogel of claim 1 or 2, wherein the radiotherapeutic hydrogel is capable of sustainably releasing the anionic radioactive agent.

4. The radiotherapeutic hydrogel of any one of claims 1-3, wherein the radiotherapeutic hydrogel can be used for selective internal radiation therapy (SIRT) via local (e.g., intratumor or intracavity) administration.

5. The radiotherapeutic hydrogel of any one of claims 1-4, wherein the biopolymer hydrogel comprises Pluronic™ F-127, methylcellulose and/or chitosan.

6. The radiotherapeutic hydrogel of any one of claims 1-5, wherein the biopolymer hydrogel further comprises a thermosensitive polymer, a pH-sensitive polymer, a crosslinker, or a combination thereof.

7. The radiotherapeutic hydrogel of any one of claims 1-6, wherein the biopolymer hydrogel further comprises a radiolysis stabilizer.

8. The radiotherapeutic hydrogel of any one of claims 1-7, wherein the radiotherapeutic hydrogel comprises a cationic biopolymer-based NP or MS, wherein the cationic biopolymer-based NP or MS is synthesized by crosslinking cationic biopolymers (e.g., chitosan) using an anionic polymer, an ionic crosslinker, a covalent crosslinker, or a combination thereof.

9. The radiotherapeutic hydrogel of any one of claims 1-8, wherein the anionic radioactive agent is selected from:
a) phosphate P-32 (e.g., H₃PO₄, H₂PO₄⁻, HPO₄²⁻, or PO₄³⁻), ATP P-32 (adenosine-5′-triphosphate), IUdR I-125 (5-iodo-2-deoxyuridine);
b) anionic radioisotopes (e.g., Astatine-211, Iodine-125, or Iodine-131); and
c) anionic forms of chelate-radiometal compounds.

10. The radiotherapeutic hydrogel of any one of claims 1-9, wherein the anionic radioactive agent possesses high cytotoxicity and/or selectivity to proliferating tumor cells.

11. The radiotherapeutic hydrogel of any one of claims 1-10, wherein the radiotherapeutic hydrogel is used for selective internal radiation therapy (SIRT) via an intratumor administration.

12. The radiotherapeutic hydrogel of claim 11, wherein the intratumor administration comprises imaging-guided percutaneous and/or intraoperative injection.

13. The radiotherapeutic hydrogel of any one of claims 1-10, wherein the radiotherapeutic hydrogel is used for selective internal radiation therapy (SIRT) via an intracavity administration.

14. The radiotherapeutic hydrogel of claim 13, wherein the intracavity administration comprises administering into peritoneal cavity, thoracic cavity, a postsurgical cavity, and/or a rescission site of a solid tumor, via a catheter impregnation and/or a direct injection.

15. A method of making a radiotherapeutic hydrogel, comprising:
a) associating an anionic radioactive agent with a cationic biopolymer, forming a radioactive biopolymer; and
b) dispersing and/or immobilizing the radioactive biopolymer into a biopolymer hydrogel.

16. A method of making a radiotherapeutic hydrogel, comprising:
a) encapsulating an anionic radioactive agent into a cationic biopolymer-based nanoparticle (NP) or microsphere (MS), forming a radioactive biopolymer-based NP or MS; and
b) dispersing and/or immobilizing the radioactive biopolymer-based NP or MS into a biopolymer hydrogel.

17. The method of claim 15 or 16, wherein the method further comprises formulating the biopolymer hydrogel for injection and/or implantation.

18. The method of any one of claims 15-17, wherein the anionic radioactive agent is selected from:
a) phosphate P-32 (e.g., H₃PO₄, H₂PO₄⁻, HPO₄²⁻, or PO₄³⁻), ATP P-32 (adenosine-5′-triphosphate), IUdR 1-125 (5-iodo-2-deoxyuridine);
b) anionic radioisotopes (e.g., Astatine-211, Iodine-125, or Iodine-131); and
c) anionic forms of chelate-radiometal compounds.

19. The method of any one of claims 15-18, wherein the anionic radioactive agent (e.g., phosphate P-32) possesses high cytotoxicity and/or selectivity to proliferating tumor cells.

20. The method of any one of claims 16-19, wherein the cationic biopolymer-based NP or MS is synthesized by crosslinking cationic biopolymers (e.g., chitosan) using an anionic polymer, a crosslinker, or a combination thereof.

21. The method of claim 20, wherein the crosslinker is an ionic crosslinker, a covalent crosslinker, or a combination thereof.

22. The method of any one of claims 15-21, wherein the biopolymer hydrogel comprises Pluronic™ F-127, methylcellulose and/or chitosan.

23. The method of any one of claims 15-22, wherein the biopolymer hydrogel further comprises a thermosensitive polymer, a pH-sensitive polymer, a crosslinker, or a combination thereof, for properly controlling the gelation of the hydrogel at near physiological pH, e.g., pH 7.2±0.2, and temperature, e.g., 36.5±1.0° C.

24. The method of any one of claims 15-23, wherein the biopolymer hydrogel further comprises a radiolysis stabilizer.

25. A method for treating a subject having cancer, the method comprising administering a therapeutically effective amount of the radiotherapeutic hydrogel of any one of claims 1-14 to the subject.

26. The method of claim 25, wherein the subject has a solid tumor.

27. The method of claim 25 or 26, wherein the radiotherapeutic hydrogel as a new approach of selective internal radiation therapy (SIRT) is administered via local (e.g., intratumor or intracavity) administration.

28. The method of claim 27, wherein the radiotherapeutic hydrogel is administered through an intratumor administration, wherein the intratumor administration comprises imaging-guided percutaneous and/or intraoperative injection.

29. The method of claim 27, wherein the radiotherapeutic hydrogel is administered into peritoneal cavity, thoracic cavity, a postsurgical cavity, and/or a rescission site of a solid tumor, via a catheter impregnation and/or a direct injection.

30. The method of any one of claims 25-29, wherein the radiotherapeutic hydrogel is biodegradable in vino over time.

31. The method of any one of claims 25-30, wherein the radiotherapeutic hydrogel sustainably releases the anionic radioactive agent.

32. The method of claim 31, wherein the sustainably released anionic radioactive agent effectively eradicates the satellite tumor cells surrounding the loading sites.

33. The method of any one of claims 25-32, wherein the anionic radioactive agent (e.g., phosphate P-32) possesses high cytotoxicity and/or selectivity to proliferating tumor cells.

34. The method of claim any one of claims 25-33, wherein the radioactivity retained within the administrated hydrogel delivers localized radiotherapy as ionizing irradiation to the region of interest as micro-brachytherapy.

35. The method of any one of claims 25-34, wherein the biopolymer hydrogel comprises Pluronic™ F-127, methylcellulose and/or chitosan.

36. The method of any one of claims 25-35, wherein the radiotherapeutic hydrogel comprises the cationic biopolymer-based NP or MS associated with the anionic radioactive agent that is dispersed into the hydrogel, wherein the cationic biopolymer-based NP or MS is synthesized by crosslinking cationic biopolymers (e.g., chitosan) using an anionic polymer, an ionic crosslinker, a covalent crosslinker, or a combination thereof.
